(19) United States
(12) Patent Application Publication
(10) Pub. No.: US 2022/0348877 A1
(43) Pub. Date: Nov. 3, 2022

(54) GENERATION OF QUIESCENT CARDIAC FIBROBLASTS FROM HUMAN INDUCED PLURIPOTENT STEM CELLS FOR IN VITRO MODELING OF CARDIAC FIBROSIS

(71) Applicant: The Board of Trustees of the Leland Stanford Junior University, Stanford, CA (US)

(72) Inventors: Joseph C. Wu, Stanford, CA (US); Hao Zhang, Stanford, CA (US)

(21) Appl. No.: 17/621,453

(22) PCT Filed: Jun. 26, 2020
(86) PCT No.: PCT/US2020/039840

§ 371 Date: Dec. 21, 2021

Related U.S. Application Data
(60) Provisional application No. 62/868,760, filed on Jun. 28, 2019.

(51) Int. Cl. C12N 5/077

(52) U.S. Cl. CPC C12N 5/0656; C12N 5/0657; C12N 2501/115; C12N 2503/02; C12N 2506/45; C12N 2501/15

(57) ABSTRACT

Human cardiac fibroblasts obtained from induced pluripotent stem cells (iPS cells) are provided for use in analysis, screening programs, and the like.

### CROSS REFERENCE

This application claims benefit of U.S. Provisional Patent Application No. 62/868,760, filed Jun. 28, 2019, which applications are incorporated herein by reference in their entirety.

### GOVERNMENT SUPPORT

This invention was made with Government support under contract HL141371, awarded by the National Institutes of Health. The Government has certain rights in the invention.

### BACKGROUND

Fibroblasts are highly plastic and can transdifferentiate into myofibroblasts after tissue injury. Myofibroblasts are distinct from quiescent fibroblasts by demonstrating a contractile phenotype with a greater synthetic ability to produce extracellular matrix (ECM) proteins. Excessive deposition of ECM, a hallmark of tissue fibrosis, can take place in various organs, and may ultimately lead to organ failure. Because the mechanisms underlying tissue fibrosis remain poorly understood, effective antifibrosis therapeutic strategies are not yet available. However, accumulating evidence suggests that the regulatory networks governing fibrogenesis are tissue-specific. Therefore, it would be more effective to develop tissue-specific anti-fibrotic therapies rather than “one-size-fits-all” drugs. However, gene markers that identify fibroblasts from various organs are largely undetermined, making it challenging to delineate the mechanisms of fibrosis in a tissue-specific manner.

As an emerging and powerful tool for comprehensive transcriptome profile analysis, single cell RNA-sequencing (scRNA-seq) technology has provided profound insights into the diversity of cell populations, the heterogeneity of gene expression patterns within the same cell type, and the complex developmental trajectories of different cell types. This powerful technology may help uncover tissue-specific transcriptome profiles of fibroblasts.

Due to the limited availability of primary human cells and challenges to propagate them long term in vitro, as well as the limited translational value of animal models in recapitulating phenotypic hallmarks of diseases in humans, human induced pluripotent stem cell (iPSC) technology has been increasingly used for disease modeling and drug screening. However, until now a reliable protocol to generate human iPSC-derived cardiac fibroblasts has not been available.

Pharmaceutical drug discovery, a multi-billion dollar industry, involves the identification and validation of therapeutic targets, as well as the identification and optimization of lead compounds. The explosion in numbers of potential new targets and chemical entities resulting from genomics and combinatorial chemistry approaches over the past few years has placed enormous pressure on screening programs. The rewards for identification of a useful drug are enormous, but the percentages of hits from any screening program are generally very low. Desirable compound screening methods solve this problem by both allowing for a high throughput so that many individual compounds can be tested; and by providing biologically relevant information so that there is a good correlation between the information generated by the screening assay and the pharmaceutical effectiveness of the compound.

Some of the more important features for pharmaceutical effectiveness are specificity for the targeted cell or disease, a lack of toxicity at relevant dosages, and specific activity of the compound against its molecular target. The present invention addresses this issue.

### SUMMARY

Compositions and methods are provided for differentiation and screening of cardiac fibroblasts for therapeutic drugs and treatment regimens, where the methods utilize in vitro cell cultures or animal models derived therefrom. Diseases of particular interest include cardiac fibrosis. The methods differentiate cardiac fibroblasts from human pluripotent stem cells, including without limitation induced pluripotent stem cells (iPS cells), which may be obtained from patient or carrier cell samples, e.g. adipocytes, fibroblasts, and the like. In some embodiments human iPSCs are induced to become cardiac fibroblasts (CF), particularly quiescent cardiac fibroblasts. In some embodiments the CF are co-cultured with primary or in vitro-generated cardiac myocytes.

Benefits of the in vitro generated CF include a high level of purity for cells with a high level of similarity to the corresponding primary cells at both genetic and functional levels, and the ability to preserve a quiescent phenotype over multiple passages. The in vitro generated CF are sensitive to pro-fibrotic stimuli, and are demonstrated to be highly responsive to both anti- and pro-fibrotic drugs. The in vitro generation of CF can be scaled up to generate large numbers of CFs for mechanistic studies and high throughput anti-fibrotic drug discovery. The induction protocol optionally provides for testing of sex- and patient-specific cellular responses to candidate anti-fibrotic drugs in vitro.

The in vitro differentiation protocol for CF utilizes a series of steps in specific media. Initially human pluripotent stem cells are differentiated to pluripotent stem cell-derived epicardial cells (iPSC-EPCs); which epicardial cells are then differentiated to cardiac fibroblasts.

In some embodiments, epicardial cells are differentiated from iPSC by (i) culture in medium comprising an effective dose of a wnt agonist, (ii) recovery for a period of time, (iii) culture in medium comprising an effective dose of a wnt inhibitor, (iv) replating in medium in the absence of these factors. After about 1 week, the cells are (v) treated with an effective dose of a wnt activator and retinoic acid, before a final recovery period.

In some embodiments, the induced epicardial cells are differentiated to CF by culture is medium comprising an effective dose of FGF2 and an inhibitor of TGFβ. Inhibiting the TGF-β pathway during differentiation can preserve the fibroblast quiescent phenotype and suppress transdifferentiation. Notably, most of these cells remained quiescent after several passages, and provide a reliable cell model for the fibrosis studies in vitro. After about 4, about 6, about 10, about 12, about 14, about 16, about 18 or more days of extended culture, for example about day 18 of differentiation, the cells show a typical fibroblast morphology and express markers (GATA4 and DDR2) that are highly expressed in primary CFs. The human iPSC-CFs are highly pure cultures and can preserve cardiac fibroblast identity during serial passaging.

In some embodiments, in vitro cell cultures of cardiac fibroblasts are provided, which may be quiescent human cardiac fibroblasts (CF). The CF may be highly pure, e.g. at least about 75% of the cells in culture, at least about 80%, at least about 85%, at least about 90% or more of the cells in a culture have fibroblast morphology and are GATA4⁺ DDR2⁺.

CFs are capable of interacting with a broad spectrum of cell types, including cardiac myocytes (CMs), endothelial cells, cardiac smooth muscle (CSM) cells, and immune cells through different signaling pathways. In some embodiments in vitro cell cultures comprise a defined co-culture of human cardiac fibroblasts a second, interacting cell type. In some such embodiments, a co-culture comprises a defined ratio of human cardiac fibroblasts and one or both of cardiac smooth muscle (CSM) cells and cardiac myocytes (CM). The ratio of CF:CSM or CF:CM may be from about 50:1, 20:1, 10:1, 5:1, 2:1, 1:1, 1:2, 1:5, 1:10, 1:20, 1:50. In some embodiments, co-culture of one or more of such cells are used in screening. It is shown that in co-cultures of CM and Cs, there is cross-talk that can play a role in suppressing cardiac fibrosis, for example through the ANP/BNP pathway. Anti-fibrotic effects seen in co-culture can be enhanced by inhibiting the activity of ANP/BNP-degrading enzyme, neprilysin.

In some embodiments of the invention, methods are provided for determining the activity of a candidate agent on a cardiac fibroblast, the method comprising contacting the candidate agent with one or a panel of in vitro generated cardiac fibroblasts as described herein, or a co-culture comprising one or a panel of in vitro generated cardiac fibroblasts; and determining the effect on a phenotype of the CF. In some embodiments the phenotype is development of fibrosis. In some embodiments screening includes testing of sex- and patient-specific cellular responses to candidate anti-fibrotic drugs in vitro.

### BRIEF DESCRIPTION OF THE DRAWINGS

FIG. 1. Mouse single-cell transcriptome reveals tissue-specific gene markers for fibroblasts are conserved in humans. A, A t-SNE (t-distributed stochastic neighbor embedding) plot showing the distribution patterns of 4,685 fibroblasts derived from 10 tissues of healthy adult mice. The numbers of tissue-specific fibroblasts used for transcriptome analysis are listed in the brackets next to the individual tissue types. B, Representative t-SNE plots showing tissue-specific fibroblast subpopulations express genes reported to be detected in fibroblasts with high abundance. C, Gene ontology (GO) enrichment analysis reveals that all the cell clusters in (A) possess fibroblast-specific biological functions. D, A heatmap comparing the most specifically expressed (25% fibroblasts expressed, log FC≥1.5, and FDR adjusted P-value<1%; Wilcoxon rank sum test) transcription factors in 10 tissue-specific mouse fibroblast subpopulations. E, Cross-species validation of cardiac (GATA4 and TBX20), lung (HOXA5 and TBX4), and bladder (HOXA1 and ISL1) specific genes in human primary cardiac, lung, and bladder fibroblasts. Primary skin fibroblasts are used as a negative control. N.D., not detected. Data are presented as mean t SEM.

FIG. 2. Generation human induced pluripotent stem cell-derived cardiac fibroblasts. A, Developmental pathway enrichment analysis (FDR adjusted P-value<1%; Hypergeometric test) showing tissue-specific transcription factors (25% fibroblasts, log FC≥log 2 and FDR adjusted P value<1%; Wilcoxon rank sum test) in fibroblasts of different tissue origins. B, A schematic diagram showing the protocol for small molecule-directed differentiation from human iPSCs to iPSC-CFs. iPSC, induced pluripotent stem cell; CPC, cardiac progenitor cell; EPC, epicardial cell; CF, cardiac fibroblast; FGF2, fibroblast growth factor-2; TGF-β, transforming growth factor-β. C, Representative bright-field images showing stage-specific cell morphology changes during the 23 differentiation of human iPSC-CFs. Scale bars, 200 μm. D, Representative immunofluorescent images showing stage-specific cells express pluripotency genes (NANOG and SSEA4), cardiac mesodermal gene (NKX2-5), epicardial genes (ZO1 and WT1), and CF genes (GATA4 and DDR2) during differentiation. Scale bars, 100 μm. E, Representative flow cytometric histograms showing protein levels of COL-I, DDR2, and TCF21 in human iPSC-CFs on day 18 of differentiation (red) versus undifferentiated iPSCs (blue). F, The expression levels of cardiac-specific, early development-related genes (GATA4, TBX20, NKX2-5, and TBX5) at individual differentiation stages. N.D., not detected. G, The expression levels of epicardial markers (WT1, TBX18, and TCF21) at individual differentiation stages of iPSC-CFs. H, The expression levels of genes that are highly expressed in fibroblasts (COL1A1, DDR2, POSTN, and VIM) in stage-specific cells during iPSC-CF differentiation. I, The expression levels of lung-specific genes (TBX4 and HOXA5) in stage-specific cells during iPSC-CF differentiation. Primary LFs (lung fibroblasts) are used as a positive control. Data in (E-I) were generated based on three independent differentiations. Data are presented as mean±SEM.

FIG. 3. Human iPSC-derived cardiac fibroblasts are highly pure and preserve their cardiac identity during passaging. A, Representative immunofluorescent images showing human iPSC-CFs are positive for gene makers (COL-I, DDR2, VIM, and POSTN) that are highly expressed in primary CFs. Scale bars, 100 μm. iPSC, induced pluripotent stem cell; CF, cardiac fibroblast. B. Representative immunofluorescent images showing the expression levels of cardiac specific genes (GATA4, TBX20, and TCF21) in human iPSC-CFs, primary cardiac and skin fibroblasts. SF, skin fibroblast. Scale bars, 100 μm. C, The expression levels of cardiac-specific genes (GATA4, TBX20, and TCF21) in human iPSC-CFs at different passages. D, The expression 24 levels of epicardial marker genes (WT1 and TBX18) in human iPSC-EPCs and iPSC-CFs at different passages. EPC, epicardial cell. E, Representative flow cytometric histograms showing the percentage of WT1+ population in human iPSC-EPCs and iPSC-CFs at different passages. Primary CFs are used as a control. The quantitative data are shown on the right. F, Representative immunofluorescent images showing negligible expression of SMC (smooth muscle cell)-specific genes (ACTA2, CNN1, SMTN, and TAGLN) in iPSC-CFs. The same iPSC lines were used to derive SMCs of the epicardial lineage, which are used as a positive control. Scale bars, 100 μm. G. The expression levels of SMC-specific genes (ACTA2, CNN1, MYH11, and TAGLN) in human iPSCCFs versus iPSC-SMCs of the epicardial lineage. H, The expression levels of pericyte-specific genes (PDGFRB, CSPG4, and MCAM) in human iPSC-CFs, primary cardiac fibroblasts, and brain pericytes. Data in (C, D, E, G, and H) are generated from three independent iPSC lines, and are presented as mean±SEM. *P<0.05 by one-way ANOVA followed by Bonferroni multiple comparisons.

FIG. 4. Human iPSC-derived cardiac fibroblasts demonstrate similar biological characteristics to primary cardiac fibroblasts. A, Expression levels of cardiac-specific genes (GATA4, TBX20, and TCF21) in human primary CFs in the absence or presence of TGF-β±a TGF-β inhibitor SB431542 (SB). CF, cardiac fibroblast. *P<0.05 vs. vehicle control, #P<0.05 vs. TGF-β group by one-way ANOVA followed by Bonferroni multiple comparisons. B, Expression levels of cardiac-specific genes (GATA4, TBX20, and TCF21) in human iPSC-CFs in the absence or presence of TGF-β±SB. iPSC, induced pluripotent stem cell. *P<0.05 vs. vehicle control, #P<0.05 vs. TGF-β group by one-way ANOVA followed by Bonferroni multiple comparisons. C, Secreted collagen contents (measured by Sirius red) are increased in both human iPSC-CFs and 25 primary CFs after TGF-β treatment. Human iPSCs were used as a negative control. *P<0.05 by one-way ANOVA followed by Bonferroni multiple comparisons. D, Representative collagen gel contraction responses in two independent human iPSC-CFs in the absence or presence of TGF-β±SB. The residual surface areas of collagen gel in each group were used for quantification (shown below the representative image), and normalized to that of the vehicle control. The size of the collagen gel area is inversely proportional to the contractility. *P<0.05 by one-way ANOVA followed by Bonferroni multiple comparisons. E, Representative images showing human iPSC derived and primary CF migration in the absence or presence of PDGF-BB (25 ng/ml) at different time points over the 24-hour observation frame using a wound-healing assay. Quantitative data showing similar migration rate (wound closure area) between human iPSC-CFs and primary CFs under the same conditions. *P<0.05 indicates PDGF-BB treatment vs. vehicle control in primary CF, #P<0.05 indicates PDGF-BB treatment vs. vehicle control in iPSC-CF, by one-way ANOVA followed by Bonferroni multiple comparisons. All data were generated from three or four independent iPSC lines, and are presented as mean±SEM.

FIG. 5. Human iPSC-derived cardiac fibroblasts are quiescent fibroblasts and therefore provide an ideal in vitro platform to model fibrosis. A-B, Representative immunofluorescent (A) and flow cytometry (B) images showing the degree of spontaneous transdifferentiation of iPSC-CFs to myofibroblasts (α-SMA+, smooth muscle α-actin) during passages 1-5. TGF-β treated CFs were used as a positive control. The percentage of SMA+ myofibroblasts in each passage of iPSC-CFs are calculated based on the flow cytometry (B, right). iPSC, induced pluripotent stem cell; CF, cardiac fibroblast. Scale bars, 100 μm. C, The expression levels of myofibroblast markers (ACTA2 and POSTN) in human iPSC-CFs cultured in the absence or presence of TGF-β±a TGF26 β inhibitor SB431542 (SB). *P<0.05 vs. vehicle control, #P<0.05 vs. TGF-β group by one-way ANOVA followed by Bonferroni multiple comparisons. D, A representative immunoblot showing the α-SMA levels in CFs derived from two independent iPSC lines in the absence or presence of TGF-β±SB. The corresponding densitometric quantification data are shown next to the immunoblot. *P<0.05 by one-way ANOVA followed by Bonferroni multiple comparisons. E-H, The expression levels of genes coding extracellular matrix proteins (collagen, fibronectin, proteoglycans, integrins, and matrix proteinases and inhibitors) in human iPSC-CFs cultured in the absence or presence of TGF-β. *P<0.05 vs. vehicle control by one-way ANOVA followed by Bonferroni multiple comparisons. I, Representative flow cytometry images and quantitative data showing pirfenidone (a commercial anti-fibrosis drug) or SB significantly suppresses TGF-β induced propagation of the α-SMA+ population in iPSC-CFs. PFD, pirfenidone. *P<0.05 vs. TGF-β group by one-way ANOVA followed by Bonferroni multiple comparisons. J, Representative flow cytometric histograms and quantitative data showing a dose-dependent induction of the α-SMA+ population after human iPSC-CFs were treated with doxorubicin. *P<0.05 vs. ctrl group by one-way ANOVA followed by Bonferroni multiple comparisons. Data in (B-J) were generated from three independent iPSC lines, and are presented as mean±SEM.

FIG. 6. Human iPSC-derived cardiomyocytes attenuate TGF-β-induced fibrotic responses in iPSC-derived cardiac fibroblasts through a paracrine mechanism. A, Computational analysis showing broad intercellular communications between fibroblasts and a diverse array of cell types through different complementary ligand-receptor signaling pathways. The threshold for defining a ligand/receptor expressed in a cluster is ≥25% with gene expression >0. FB, fibroblast. B, The basal expression levels of ligands NPPA (natriuretic peptide type A) and NPPB and their 27 cognate receptor NPR1 (natriuretic peptide receptor 1) in human iPSC-CMs and iPSC-CFs. iPSC, induced pluripotent stem cell; CM, cardiomyocyte; CF. cardiac fibroblast. C, Expression levels of CF and myofibroblast markers in human iPSC-CFs in the absence or presence of TGF-β±BNP. *P<0.05 vs. vehicle control, #P<0.05 vs. TGF-β group by one-way ANOVA followed by Bonferroni multiple comparisons. D, A representative immunoblot showing α-SMA (smooth muscle α-actin) levels in human iPSC-CFs treated with TGF-β and escalating concentrations of BNP. Densitometric quantifications are shown above the immunoblot. *P<0.05 by one-way ANOVA followed by Bonferroni multiple comparisons. E, Expression levels of CF and myofibroblast markers in human iPSC-CFs after they were indirectly co-cultured with or without iPSC-CMs in the absence or presence of TGF-β±sacubitril. Sacubitril is an inhibitor for ANP (atrial natriuretic peptide) and BNP-degrading enzyme. *P<0.05 vs. vehicle control, #P<0.05 vs. TGF-β group, $P<0.05 vs. iPSC-CM co-culture group by one-way ANOVA followed by Bonferroni multiple comparisons. F, Representative immunoblots showing the levels of α-SMA, periostin (POSTN), collagen III (COLIII), and fibronectin (FN) in human iPSC-CFs after they were indirectly co-cultured with or without iPSC-CMs in the absence or presence of TGF-β±sacubitril. Densitometric quantifications are shown below the immunoblots. *P<0.05 by one-way ANOVA followed by Bonferroni multiple comparisons. G, Schematic of the proposed mechanisms for the role of cardiomyocytes in suppressing TGF-β-induced CF transdifferentiation through the ANP/BNP-NPR1 signaling pathway. Sacubitril, a pharmacological drug, can further enhance the anti-fibrotic effect by suppressing the activity of ANP- and BNP-degrading enzyme, neprilysin. Data in (B-F) were generated based on three independent differentiations, and are presented as mean±SEM.

FIG. 7. A logical flow diagram demonstrates the process of developing the human iPSC derived cardiac fibroblast differentiation protocol. The differentiation protocol for human iPSC-CFs developed in this study is based on the recognition of tissue-specific marker genes expressing in fibroblast subpopulations, as revealed by the mouse single-cell transcriptomic data. Further DEG (differentially expressed genes) analysis highlights the critical roles of tissue-specific transcription factors in regulating the development trajectories of fibroblast subpopulations, and also suggests that CFs may be derived from the epicardial lineage. As such, human iPSC-CFs were successfully generated after intermediate cell types of cardiac progenitor cells and epicardial cells being sequentially differentiated. iPSC, induced pluripotent stem cell; CF, cardiac fibroblast.

FIG. 8. Generation tissue-specific fibroblast subpopulations from Tabula Muris. A, Originally assigned annotations to fibroblast-containing cell clusters derived from different tissues and organs of healthy adult mice in the Tabula Muris database. B, A t-SNE (t-distributed stochastic neighbor embedding) plot showing the distribution pattern of the cell clusters in (A). C, t-SNE plots showing cells positive for Col1a2, Ddr2, Dcn, TiMp2, and Pdgfra (highlighted in purple) in each cluster. D, t-SNE plots showing cells positive for endothelial markers (Pecam1 and Cdh5), smooth muscle cell markers (Tagln and Cnn1), or an immune cell marker (Ptprc).

FIG. 9. Commonly expressed genes in tissue-specific fibroblast subpopulations. These genes are detected by comparing all generated tissue-specific fibroblast subpopulations with other cell types with the cutoff: ≥25% fibroblasts expressed, log FC≥log 2, FDR adjusted P-value<1% under the Wilcox rank sum test.

FIG. 10. Donor and sex effects on tissue-specific fibroblast clustering. A, A t-SNE (t-distributed stochastic neighbor embedding) plot showing a donor-independent distribution pattern of tissue-specific fibroblasts from different mice (n=4 for M and 4 for F). F, female; M, male. B, A series of t-SNE plots showing the distribution patterns of tissue specific fibroblasts in various organs of male versus female mice.

FIG. 11. Genes in fibroblasts from the heart and fat tissues showing a sex dependent expression pattern. A, A heatmap comparing top 10 specifically expressed genes in cardiac fibroblasts from male versus female mice. B, A heatmap comparing top 10 specifically expressed genes in fibroblasts of fat tissues from male versus female mice. C, Violin plots showing the expression levels of estrogen receptor (Esr1) and androgen receptor (Ar) among fibroblasts of various tissue origins.

FIG. 12. The most specifically expressed genes in tissue-specific fibroblasts. The numbers of tissue-specific fibroblasts used to generate this heatmap are 314 (bladder), 1444 (heart), 220 (lung), 411 (trachea), 19 (pancreas), 194 (limb muscle), 206 (diaphragm), 1523 (fat), 323 (mammary gland), and 31 (aorta).

FIG. 13. Expression levels of epicardial genes in mouse cardiac fibroblasts. t-SNE (t-distributed stochastic neighbor embedding) plots showing the distribution patterns of epicardial markers (Wt1, Tbx18, and Tcf21) in adult mouse cardiac fibroblasts (highlighted in purple). These data are generated from the Tabula Muris dataset.

FIG. 14. Expression levels of myogenic repressor genes in human iPSC derived cardiac fibroblasts and smooth muscle cells. Gene expression levels of myogenic repressor genes (ELK1 and KLF4) in human iPSC-derived cardiac fibroblasts versus smooth muscle cells of the epicardial lineage. iPSC, induced pluripotent stem cell; CF, cardiac fibroblast; SMC, smooth muscle cell. *P<0.05 by one-way ANOVA followed by Bonferroni multiple comparisons. Data were generated based on three independent differentiations, and are presented as mean±SEM.

FIG. 15. Human iPSC-derived cardiac fibroblasts upregulate the expression of MYH7 in iPSC-derived cardiomyocytes in the direct contact coculture condition. A representative immunoblot showing the levels of MHY7 (myosin heavy chain 7) in human iPSC-derived cardiomyocytes after cocultured with or without iPSC-derived CFs in the absence or presence of TGF-β. Densitometric quantitifactions are shown above the immunoblots. Data were generated based on three independent differentiations, and are presented as mean±SEM.

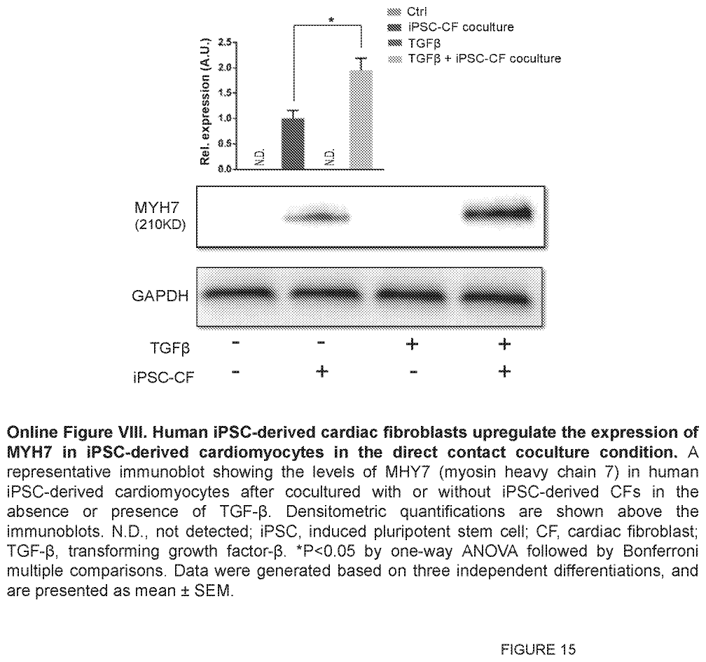

The invention is best understood from the following detailed description when read in conjunction with the accompanying drawings.

### DETAILED DESCRIPTION OF THE INVENTION

Before the present compositions and methods are described, it is to be understood that this invention is not limited to particular compositions and methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a reprogramming factor polypeptide” includes a plurality of such polypeptides, and reference to “the induced pluripotent stem cells” includes reference to one or more induced pluripotent stem cells and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

By “pluripotency” and pluripotent stem cells it is meant that such cells have the ability to differentiate into all types of cells in an organism. The term “induced pluripotent stem cell” encompasses pluripotent cells, that, like embryonic stem (ES) cells, can be cultured over a long period of time while maintaining the ability to differentiate into all types of cells in an organism, but that, unlike ES cells (which are derived from the inner cell mass of blastocysts), are derived from differentiated somatic cells, that is, cells that had a narrower, more defined potential and that in the absence of experimental manipulation could not give rise to all types of cells in the organism. iPS cells have an hESC-like morphology, growing as flat colonies with large nucleo-cytoplasmic ratios, defined borders and prominent nuclei. In addition, iPS cells express one or more key pluripotency markers known by one of ordinary skill in the art, including but not limited to alkaline phosphatase, SSEA3, SSEA4, Sox2, Oct3/4, Nanog, TRA160, TRA181, TDGF 1, Dnmt3b, FoxD3, GDF3, Cyp26a1, TERT, and zfp42. In addition, the iPS cells are capable of forming teratomas. In addition, they are capable of forming or contributing to ectoderm, mesoderm, or endoderm tissues in a living organism.

As used herein, “reprogramming factors” refers to one or more, i.e. a cocktail, of biologically active factors that act on a cell to after transcription, thereby reprogramming a cell to multipotency or to pluripotency. Reprogramming factors may be provided to the cells, e.g. cells from an individual with a family history or genetic make-up of interest for heart disease such as fibroblasts, adipocytes, etc.; individually or as a single composition, that is, as a premixed composition, of reprogramming factors. The factors may be provided at the same molar ratio or at different molar ratios. The factors may be provided once or multiple times in the course of culturing the cells of the subject invention. In some embodiments the reprogramming factor is a transcription factor, including without limitation, Oct3/4; Sox2; Klf4; c-Myc; Nanog; and Lin-28.

Somatic cells are contacted with reprogramming factors, as defined above, in a combination and quantity sufficient to reprogram the cell to pluripotency. Reprogramming factors may be provided to the somatic cells individually or as a single composition, that is, as a premixed composition, of reprogramming factors. In some embodiments the reprogramming factors are provided as a plurality of coding sequences on a vector.

Genes may be introduced into the somatic cells or the iPS cells derived therefrom for a variety of purposes, e.g. to replace genes having a loss of function mutation, provide marker genes, etc. Alternatively, vectors are introduced that express antisense mRNA or ribozymes, thereby blocking expression of an undesired gene. Other methods of gene therapy are the introduction of drug resistance genes to enable normal progenitor cells to have an advantage and be subject to selective pressure, for example the multiple drug resistance gene (MDR), or anti-apoptosis genes, such as bcl-2. Various techniques known in the art may be used to introduce nucleic acids into the target cells, e.g. electroporation, calcium precipitated DNA, fusion, transfection, lipofection, infection and the like, as discussed above. The particular manner in which the DNA is introduced is not critical to the practice of the invention.

The cells are harvested at an appropriate stage of development, which may be determined based on the expression of markers and phenotypic characteristics of the desired cell type e.g. at from about 1 to 4 weeks. Cultures may be empirically tested by staining for the presence of the markers of interest, by morphological determination, etc. The cells are optionally enriched before or after the positive selection step by drug selection, panning, density gradient centrifugation, etc. In another embodiment, a negative selection is performed, where the selection is based on expression of one or more of markers found on ES cells, fibroblasts, epithelial cells, and the like. Selection may utilize panning methods, magnetic particle selection, particle sorter selection, and the like.

The term “population”, e.g., “cell population” or “population of cells”, as used herein means a grouping (i.e., a population) of two or more cells that are separated (i.e., isolated) from other cells and/or cell groupings. For example, a 6-well culture dish can contain 6 cell populations, each population residing in an individual well. The cells of a cell population can be, but need not be, clonal derivatives of one another. A cell population can be derived from one individual cell. For example, if individual cells are each placed in a single well of a 6-well culture dish and each cell divides one time, then the dish will contain 6 cell populations. The cells of a cell population can be, but need not be, derived from more than one cell, i.e. non-clonal. The cells from which a non-clonal cell population may be derived may be related or unrelated and include but are not limited to, e.g., cells of a particular tissue, cells of a particular sample, cells of a particular lineage, cells having a particular morphological, physical, behavioral, or other characteristic, etc. A cell population can be any desired size and contain any number of cells greater than one cell. For example, a cell population can be 2 or more, 10 or more, 100 or more, 1,000 or more, 5,000 or more, 10⁴ or more, 10⁵ or more, 10⁸ or more, 107 or more, 10⁸ or more, 10⁹ or more, 10¹⁰ or more, 10¹¹ or more, 10¹² or more, 10¹³ or more, 10¹⁴ or more, 10¹⁵ or more, 10¹⁶ or more, 10¹⁷ or more, 10¹⁸ or more, 10¹⁹ or more, or 10²⁰ or more cells.

The terms “homogenous population”, as it relates to cell populations, refers to a cell population that is essentially pure and does not consist of a significant amount of undesired or contaminating cell types. By significant amount, in this context, is meant an amount of undesired or contaminating cell types that negatively impacts the use of the isolated desired cell population. As such, the actual amount of undesired or contaminating cells that defines a significant amount will vary and depend on the particular type of undesired or contaminating cells and/or the particular use of the desired cell type. For example, in a population of differentiated mesodermal cells used in the treatment of a subject, a significant amount of improperly differentiated contaminating cell types will be small as such cells may a high capacity to negatively impact the use of the generated desired cell population. In comparison, e.g., in a population of differentiated mesodermal cells used in the treatment of a subject, a significant amount of contaminating progenitor cells may be relatively large as such cells may have a low capacity to negatively impact the use of the generated desired cell population. In some instances, a homogenous population may refer to a highly enriched population. Levels of homogeneity will vary, as described, and may, in some instances, be greater than 60% pure, including e.g., more than 65%, more than 70%, more than 75%, more than 80%, more than 85%, more than 90%, more than 95%, more than 96%, more than 97%, more than 98%, more than 99%, more than 99.5%, more than 99.6%, more than 99.7%, more than 99.8%, and more than 99.9%.

The term “heterologous”, as it refers to a “heterologous sequence” or “heterologous nucleic acid”, means derived from a genotypically distinct entity from that of the rest of the entity to which it is being compared. For example, a polynucleotide introduced by genetic engineering techniques into a plasmid or vector derived from a different species is a heterologous polynucleotide. A promoter removed from its native coding sequence and operatively linked to a coding sequence with which it is not naturally found linked is a heterologous promoter.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

Quiescent cardiac fibroblasts, for the purposes of the present disclosure, are those fibroblasts that share the phenotype of quiescent primary cardiac fibroblasts. It has been previously observed that primary fibroblasts can undergo spontaneous transdifferentiation into myofibroblasts during in vitro culturing. Markers of activation, i.e. lack of quiescence, include expression of α-smooth muscle actin (α-SMA). Additional markers include transcriptional upregulation of ACTA2 and POSTN, which are significantly increased with activation. Because the transdifferentiation from fibroblasts to myofibroblasts is strongly associated with the dynamic changes of ECM genes, increased expression levels of ECM genes such as COL1A1, COL3A1, COL4A2, and FN1 are also indicative of activation, as are several proteoglycans (VCAN, CTGF, KAL1, TNC, and SPARC) and cell-matrix focal adhesion molecules (ITGA4, ITGA5, and ITGB5), and TIMP1, TIMP2, and TIMP3. There is a downregulation of MMP1 with activation.

Quiescent CF are therefore defined as those CF that share a transcriptional profile of primary, non-activated fibroblasts, e.g. with respect to the extracellular matrix, actin, proteoglycan, and cell-matrix focal adhesion genes.

In addition to various uses as in vitro cultured cells, the cardiac fibroblasts generated by the methods described herein may be tested in a suitable animal model. At one level, cells are assessed for their ability to survive and maintain their phenotype in vivo. Cell compositions are administered to immunodeficient animals (such as nude mice, or animals rendered immunodeficient chemically or by irradiation). Tissues are harvested after a period of regrowth, and assessed as to whether the administered cells or progeny thereof are still present, and may be phenotyped for response to a treatment of interest. Suitability can also be determined in an animal model by assessing the degree of cardiac recuperation that ensues from treatment with the differentiating cells of the invention. A number of animal models are available for such testing. For example, hearts can be cryoinjured by placing a precooled aluminum rod in contact with the surface of the anterior left ventricle wall (Murry et al., J. Clin. Invest. 98:2209, 1996; Reinecke et al., Circulation 100:193, 1999; U.S. Pat. No. 6,099,832). In larger animals, cryoinjury can be inflicted by placing a 30-50 mm copper disk probe cooled in liquid N₂ on the anterior wall of the left ventricle for approximately 20 min (Chiu et al., Ann. Thorac. Surg. 60:12, 1995). Infarction can be induced by ligating the left main coronary artery (Li et al., J. Clin. Invest. 100:1991, 1997). Injured sites are treated with cell preparations of this invention, and the heart tissue is examined by histology for the presence of the cells in the damaged area. Cardiac function can be monitored by determining such parameters as left ventricular end-diastolic pressure, developed pressure, rate of pressure rise, rate of pressure decay, etc.

The terms “individual,” “subject,” “host,” and “patient,” are used interchangeably herein and refer to any mammalian subject for whom diagnosis, treatment, or therapy is desired, particularly humans.

“Myocardial fibrosis” is the excessive deposition of extracellular matrix proteins, including collagens, in the heart. In cardiomyopathies, the formation of interstitial fibrosis and/or replacement fibrosis is almost always part of the pathological cardiac remodeling process. Different forms of cardiomyopathies show particular patterns of myocardial fibrosis that can be considered as distinctive hallmarks. Although formation of fibrosis is initially aimed to be a reparative mechanism, in the long term, on-going and excessive myocardial fibrosis may lead to arrhythmias and stiffening of the heart wall and subsequently to diastolic dysfunction. Ultimately, adverse remodeling with progressive myocardial fibrosis can lead to heart failure.

Fibrosis is a characteristic of many chronic diseases, including cardiac disease and heart failure. In case of myocardial fibrosis, the balance between synthesis and degradation of ECM proteins is disturbed, leading to excess fibrous connective tissue formation. A main event that occurs is CF activation and differentiation into myofibroblasts, which is primarily triggered by transforming growth factor (TGF-β). Myofibroblasts secrete higher amounts of ECM proteins, thereby playing a key role in fibrosis formation. Interstitial fibrosis is characterized by an increase in ECM and collagen deposits between the cells without loss of cardiomyocytes. The fibrosis is diffusely present throughout the myocardium. This type of fibrosis is mostly caused by chronic triggers, such as pressure overload (hypertension), inflammation, and aging. Perivascular fibrosis is characterized by accumulation of collagen fibres in the area surrounding the coronary arteries, and is mainly observed in the setting of hypertension. Replacement fibrosis presents following cardiomyocyte death, and occurs for instance after acute ischemic injury, such as myocardial infarction (MI).

The standard for visualization of myocardial fibrosis consists of histological analysis of tissue, for example using Masson's trichrome or Sirius red staining. Non-invasive techniques have emerged to investigate cardiac fibrosis in vivo, most commonly using cardiac magnetic resonance (CMR) imaging, e.g. late gadolinium enhancement (LGE) Normal myocardium is visualized as a “dark” area without Gd, and damaged myocardium will be seen as “bright” areas of Gd accumulation. CMR techniques using T1 mapping have also been developed. T1 mapping consists of the generation of a pixelated map based on the longitudinal or spin-lattice relaxation of protons that recover towards thermodynamic equilibrium following excitation with the radiofrequency beam. In case of myocardial fibrosis, an increase in native T1 relaxation time will be observed. T1 mapping can also be combined with administration of Gd, which increases proton relaxation, and thus decreases T1 relaxation time. Native and post-contrast T1 mapping can determine an increase in ECM volume that is not detectable by LGE.

Hypertrophic cardiomyopathy (HCM) is characterized by left ventricular hypertrophy (LVH), which cannot be explained by abnormal loading conditions. In the majority of HCM patients, often extensive amounts of fibrosis are observed. Typical to HCM, this is most commonly seen as patchy replacement fibrosis, either alone or in combination with interstitial fibrosis, and mostly present at the (basal) IVS and the attachment of the RV and LV.

Dilated cardiomyopathy (DCM) is characterized by ventricular dilatation and often progressive contractile dysfunction, resulting in great risk of developing HF. Many DCM patients present with substantial amounts of myocardial fibrosis. In these patients, myocardial fibrosis is mostly observed as replacement fibrosis with a midwall distribution throughout the LV. Similar to HCM, myocardial fibrosis is related to disease severity in DCM.

Left ventricular non-compaction (LVNC) is a very rare form of cardiomyopathy, which is characterized by structural abnormality of the LV myocardium. The etiology of LVNC is unknown but is believed to be genetic in nature, causing failure of the compaction process of the myocardial wall during development. Myocardial fibrosis may be present in the compacted myocardium, and most commonly in the IVS.

Laminopathies are caused by pathogenic variants in the lamin A/C (LMNA) gene. The lamin A and C proteins are produced via alternative splicing of this gene, and are components of the nuclear lamina and hence essential in proper nuclear architecture and function. Several autosomal dominant mutations in the LMNA gene have been associated with cardiac disease that finally can culminate in a DCM phenotype. This cardiomyopathy is, however, also characterized by early-onset atrioventricular (AV)-block, (supra)ventricular arrhythmias, and SCD even before DCM development. Localized fibrosis in the IVS may be the mechanism behind reduced septal function, AV-block, and VA in lamin A/C mutation-positive subjects.

Phospholamban (PLN) p.Arg14del induced cardiomyopathy shows a unique pattern of myocardial fibrosis. This mutation in the PLN gene, deleting the arginine on position 14 of the PLN protein, results in a severe form of cardiomyopathy with characteristics of DCM and ARVC. This cardiomyopathy is also associated with distinct fibrotic patterns with clinical importance.

### METHODS OF THE INVENTION

Methods are provided for the obtention and use of in vitro cell cultures of cardiac fibroblasts, where the cardiac fibroblasts are differentiated from induced human pluripotent stem cells (iPS cells). In some embodiments a co-culture of such cardiac fibroblasts are provided. Candidate agents include small molecules, i.e. drugs, genetic constructs that increase or decrease expression of an RNA of interest, protein expression, and the like.

Aspects of the disclosure include methods for deriving CF from pluripotent progenitor cells. What is meant by pluripotent progenitors is described herein. Pluripotent progenitors of the instant disclosure may be acquired from any convenient source, including but not limited to newly derived from a subject of interest or tissue specimen or other cellular sample, obtained from a public repository, obtained from a commercial vendor, and the like. In some instances, pluripotent cells of interest include human cells including but not limited to, e.g., human embryonic stem cells, human induced pluripotent stem cells, human fetal stem cells, and the like.

In some instances, pluripotent progenitor cells of the subject disclosure may be unmodified such that the cells have not been genetically or otherwise modified from their natural state prior to modification according the methods described herein. In other instances, pluripotent progenitor cells of the subject disclosure may be unmodified such that the cells have been genetically or otherwise modified from their natural state prior to modification according the methods described herein.

Following induction of pluripotency, hiPSC are cultured according to any convenient method, e.g. on irradiated feeder cells and commercially available medium. The hiPSC can be dissociated from feeders by digesting with protease, e.g. dispase, preferably at a concentration and for a period of time sufficient to detach intact colonies of pluripotent stem cells from the layer of feeders, or from hiPSC grown in feeder-free conditions, by dissociation into a single cell suspension and aggregation using various approaches, including centrifugation in plates, etc.

Genes may be introduced into the somatic cells or the hiPSC derived therefrom for a variety of purposes, e.g. to replace genes having a loss of function mutation, provide marker genes, etc. Alternatively, vectors are introduced that express antisense mRNA, siRNA, ribozymes, etc. thereby blocking expression of an undesired gene. Other methods of gene therapy are the introduction of drug resistance genes to enable normal progenitor cells to have an advantage and be subject to selective pressure, for example the multiple drug resistance gene (MDR), or anti-apoptosis genes, such as BCL-2. Various techniques known in the art may be used to introduce nucleic acids into the target cells, e.g. electroporation, calcium precipitated DNA, fusion, transfection, lipofection, infection and the like, as discussed above. The particular manner in which the DNA is introduced is not critical to the practice of the invention.

Disease-associated or disease-causing genotypes can be generated in healthy hiPSC through targeted genetic manipulation (CRISPR/CAS9, etc) or hIPSC can be derived from individual patients that carry a disease-related genotype or are diagnosed with a disease, e.g. Hypertrophic cardiomyopathy (HCM), dilated cardiomyopathy (DCM), left ventricular non-compaction (LVNC), laminopathies, phospholamban (PLN) p.Arg14del, etc. Moreover, cardiac diseases with less defined or without genetic components can be studied within the model system. Conditions of neurodevelopmental and neuropsychiatric disorders and neural diseases that have strong genetic components or are directly caused by genetic or genomic alterations can be modeled with the systems of the invention.

Mutations of interest include mutations in the genes: cardiac troponin T (TNNT2); myosin heavy chain (MYH7); tropomyosin 1 (TPM1); myosin binding protein C (MYBPC3); 5′-AMP-activated protein kinase subunit gamma-2 (PRKAG2); troponin I type 3 (TNNI3); titin (TTN); myosin, light chain 2 (MYL2); actin, alpha cardiac muscle 1 (ACTC1); potassium voltage-gated channel, KQT-like subfamily, member 1 (KCNQ1); plakophilin 2 (PKP2); and cardiac LIM protein (CSRP3). Specific mutations of interest include, without limitation, MYH7 R663H mutation; TNNT2 R173W; PKP2 2013delC mutation; PKP2 Q617X mutation; and KCNQ1 G269S missense mutation. Any number of disorders with a defined genetic etiology can be additionally modeled by introducing mutations in or completely removing disease-relevant gene(s) in control hiPSC using genome editing, e.g. CRISPR. A particular advantage of this method is that fact that edited hiPSC lines share the same genetic background as their corresponding, non-edited hiPSC lines. This reduces variability associated with line-line differences in genetic background.

In some embodiments a panel of such cardiomyocytes are provided, where the panel includes two or more different disease-relevant cardiomyocytes. In some embodiments a panel of such cardiomyocytes are provided, where the cardiomyocytes are subjected to a plurality of candidate agents, or a plurality of doses of a candidate agent. Candidate agents include small molecules, i.e. drugs, genetic constructs that increase or decrease expression of an RNA of interest, electrical changes, and the like. In some embodiments the disease-relevant cardiomyocytes are introduced or induced to differentiate from iPS cells in an in vivo environment, for example as an explant in an animal model. In some embodiments a panel refers to a system or method utilizing patient-specific cardiomyocytes from two or more distinct cardiac conditions, and may be three or more, four or more, five or more, six or more, seven or more distinct conditions, where the conditions are selected from: dilated cardiomyopathy (DCM); hypertrophic cardiomyopathy (HCM); anthracycline-induced cardiotoxicity; arrhythmogenic right ventricular dysplasia (ARVD); left ventricular non-compaction (LVNC); double inlet left ventricle (DILV); and long QT (Type-1) syndrome (LQT-1).

In some instances, pathway modulating agents, as described above and including pathway activators and pathway inhibitors include, e.g., those that are commercially available, e.g., from such suppliers such as Tocris Bioscience (Bristol, UK), Sigma-Aldrich (St. Louis, Mo.), Santa Cruz Biotechnology (Santa Cruz, Calif.), and the like.

Pluripotent progenitors and derivatives thereof may be contacted with induction agents by any convenient means. Generally an induction agent is added to culture media, as described herein, within which cells of the instant disclosure are grown or maintained, such that the induction agent is present, in contact with the cells, at an effective concentration to produce the desired effect, e.g., induce a desired lineage restriction event. In other instances, e.g., where the existing culture media is not compatible with a particular induction agent, the culture media in which the cells are being grown is replaced with fresh culture media containing the particular induction agent present in the fresh media at an effective concentration to produce the desired effect. In instances where fresh or specific culture media is provided with a particular induction agent the culture agent may, in some instances, be specifically formulated for the particular induction agent, e.g., containing one or more specific additional reagents to, e.g., aid in the delivery of the induction agent, aid in the solubility of the induction agent, aid in the stability of the induction agent, etc.

The effective concentration of a particular induction agent will vary and will depend on the agent. In addition, in some instances, the effective concentration may also depend on the cells being induced, the culture condition of the cells, other induction agents co-present in the culture media, etc. As such, the effective concentration of induction agents will vary and may range from 1 ng/mL to 10 μg/mL or more, including but not limited to, e.g., 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 11 ng/mL, 12 ng/mL, 13 ng/mL, 14 ng/mL, 15 ng/mL, 16 ng/mL, 17 ng/mL, 18 ng/mL, 19 ng/mL, 20 ng/mL, 21 ng/mL, 22 ng/mL, 23 ng/mL, 24 ng/mL, 25 ng/mL, 26 ng/mL, 27 ng/mL, 28 ng/mL, 29 ng/mL, 30 ng/mL, 31 ng/mL, 32 ng/mL, 33 ng/mL, 34 ng/mL, 35 ng/mL, 36 ng/mL, 37 ng/mL, 38 ng/mL, 39 ng/mL, 40 ng/mL, 41 ng/mL, 42 ng/mL, 43 ng/mL, 44 ng/mL, 45 ng/mL, 46 ng/mL, 47 ng/mL, 48 ng/mL, 49 ng/mL, 50 ng/mL, 1-5 ng/mL, 1-10 ng/mL, 1-20 ng/mL, 1-30 ng/mL, 1-40 ng/mL, 1-50 ng/mL, 5-10 ng/mL, 5-20 ng/mL, 10-20 ng/mL, 10-30 ng/mL, 10-40 ng/mL, 10-50 ng/mL, 20-30 ng/mL, 20-40 ng/mL, 20-50 ng/mL, 30-40 ng/mL, 30-50 ng/mL, 40-50 ng/mL, 1-100 ng/mL, 50-100 ng/mL, 60-100 ng/mL, 70-100 ng/mL, 80-100 ng/mL, 90-100 ng/mL, 10-100 ng/mL, 50-200 ng/mL, 100-200 ng/mL, 50-300 ng/mL, 100-300 ng/mL, 200-300 ng/mL, 50-400 ng/mL, 100-400 ng/mL, 200-400 ng/mL, 300-400 ng/mL, 50-500 ng/mL, 100-500 ng/mL, 200-500 ng/mL, 300-500 ng/mL, 400 to 500 ng/mL, 0.001-1 μg/mL, 0.001-2 μg/mL, 0.001-3 μg/mL, 0.001-4 μg/mL, 0.001-5 μg/mL, 0.001-6 μg/mL, 0.001-7 μg/mL, 0.001-8 μg/mL, 0.001-9 μg/mL, 0.001-10 μg/mL, 0.01-1 μg/mL, 0.01-2 μg/mL, 0.01-3 μg/mL, 0.01-4 μg/mL, 0.01-5 μg/mL, 0.01-6 μg/mL, 0.01-7 μg/mL, 0.01-8 μg/mL, 0.01-9 μg/mL, 0.01-10 μg/mL, 0.1-1 μg/mL, 0.1-2 μg/mL, 0.1-3 μg/mL, 0.1-4 μg/mL, 0.1-5 μg/mL, 0.1-6 μg/mL, 0.1-7 μg/mL, 0.1-8 μg/mL, 0.1-9 μg/mL, 0.1-10 μg/mL, 0.5-1 μg/mL, 0.5-2 μg/mL, 0.5-3 μg/mL, 0.5-4 μg/mL, 0.5-5 μg/mL, 0.5-6 μg/mL, 0.5-7 μg/mL, 0.5-8 μg/mL, 0.5-9 μg/mL, 0.5-10 μg/mL, and the like.

In some instances, the effective concentration of an induction agent in solution, e.g., cell culture media, may range from 1 nM to 100 μM or more, including but not limited to, e.g., 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 21 nM, 22 nM, 23 nM, 24 nM, 25 nM, 26 nM, 27 nM, 28 nM, 29 nM, 30 nM, 31 nM, 32 nM, 33 nM, 34 nM, 35 nM, 36 nM, 37 nM, 38 nM, 39 nM, 40 nM, 41 nM, 42 nM, 43 nM, 44 nM, 45 nM, 46 nM, 47 nM, 48 nM, 49 nM, 50 nM, 1-2 nM, 1-3 nM, 1-4 nM, 1-5 nM, 1-6 nM, 1-7 nM, 1-8 nM, 1-9 nM, 1-10 nM, 1.5 nM, 1.5-2 nM, 1.5-3 nM, 1.5-4 nM, 1.5-5 nM, 1.5-6 nM, 1.5-7 nM, 1.5-8 nM, 1.5-9 nM, 1.5-10 nM, 2-3 nM, 2-4 nM, 2-5 nM, 2-6 nM, 2-7 nM, 2-8 nM, 2-9 nM, 2-10 nM, 3-4 nM, 3-5 nM, 3-6 nM, 3-7 nM, 3-8 nM, 3-9 nM, 3-10 nM, 4-5 nM, 4-6 nM, 4-7 nM, 4-8 nM, 4-9 nM, 4-10 nM, 5-6 nM, 5-7 nM, 5-8 nM, 5-9 nM, 5-10 nM, 6-7 nM, 6-8 nM, 6-9 nM, 6-10 nM, 7-8 nM, 7-9 nM, 7-10 nM, 8-9 nM, 8-10 nM, 9-10 nM, 5-15 nM, 5-20 nM, 5-25 nM, 5-30 nM, 5-35 nM, 5-40 nM, 5-45 nM, 5-50 nM, 10-15 nM, 10-20 nM, 10-25 nM, 10-30 nM, 10-35 nM, 10-40 nM, 10-50 nM, 15-20 nM, 15-25 nM, 15-30 nM, 15-35 nM, 15-40 nM, 15-45 nM, 15-50 nM, 20-25 nM, 20-30 nM, 20-35 nM, 20-40 nM, 20-45 nM, 20-50 nM, 25-30 nM, 25-35 nM, 25-40 nM, 25-45 nM, 25-50 nM, 30-35 nM, 30-40 nM, 30-45 nM, 30-50 nM, 35-40 nM, 35-45 nM, 35-50 nM, 40-45 nM, 40-50 nM, 45-50 nM, 10-100 nM, 20-100 nM, 30-100 nM, 40-100 nM, 50-100 nM, 60-100 nM, 70-100 nM, 80-100 nM, 90-100 nM, 50-150 nM, 50-200 nM, 50-250 nM, 50-300 nM, 50-350 nM, 50-400 nM, 50-450 nM, 50-500 nM, 10-150 nM, 10-200 nM, 10-250 nM, 10-300 nM, 10-350 nM, 10-400 nM, 10-450 nM, 10-500 nM, 100-150 nM, 100-200 nM, 100-250 nM, 100-300 nM, 100-350 nM, 100-400 nM, 100-450 nM, 100-500 nM, 200-500 nM, 300-500 nM, 400-500 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 200-400 nM, 300-500 nM, 400-600 nM, 500-700 nM, 600-800 nM, 700-900 nM, 800 nM to 1 μM, 0.5-1 μM, 0.5-1.5 μM, 0.5-2 μM, 0.5-2.5 μM, 0.5-3 μM, 0.5-3.5 μM, 0.5-4 μM, 0.5-4.5 μM, 0.5-5 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 41 μM, 42 μM, 43 μM, 44 μM, 45 μM, 46 μM, 47 μM, 48 μM, 49 μM, 50 μM, 1-2 μM, 1-3 μM, 1-4 μM, 1-5 μM, 1-6 μM, 1-7 μM, 1-8 μM, 1-9 μM, 1-10 μM, 1.5 μM, 1.5-2 μM, 1.5-3 μM, 1.5-4 μM, 1.5-5 μM, 1.5-6 μM, 1.5-7 μM, 1.5-8 μM, 1.5-9 μM, 1.5-10 μM, 2-3 μM, 2-4 μM, 2-5 μM, 2-6 μM, 2-7 μM, 2-8 μM, 2-9 μM, 2-10 μM, 3-4 μM, 3-5 μM, 3-6 μM, 3-7 μM, 3-8 μM, 3-9 μM, 3-10 μM, 4-5 μM, 4-6 μM, 4-7 μM, 4-8 μM, 4- 9 μM, 4-10 μM, 5-6 μM, 5-7 μM, 5-8 μM, 5-9 μM, 5-10 μM, 6-7 μM, 6-8 μM, 6-9 μM, 6-10 μM, 7-8 μM, 7-9 μM, 7-10 μM, 8-9 μM, 8-10 μM, 9-10 μM, 5-15 μM, 5-20 μM, 5-25 μM, 5-30 μM, 5-35 μM, 5-40 μM, 5-45 μM, 5-50 μM, 10-15 μM, 10-20 μM, 10-25 μM, 10-30 μM, 10-35 μM, 10-40 μM, 10-50 μM, 15-20 μM, 15-25 μM, 15-30 μM, 15-35 μM, 15-40 μM, 15-45 μM, 15-50 μM, 20-25 μM, 20-30 μM, 20-35 μM, 20-40 μM, 20-45 μM, 20-50 μM, 25-30 μM, 25-35 μM, 25-40 μM, 25-45 μM, 25-50 μM, 30-35 μM, 30-40 μM, 30-45 μM, 30-50 μM, 35-40 μM, 35-45 μM, 35-50 μM, 40-45 μM, 40-50 μM, 45-50 μM, 10-100 μM, 20-100 μM, 30-100 μM, 40-100 μM, 50-100 μM, 60-100 μM, 70-100 μM, 80-100 μM, 90-100 μM, and the like.

In some instances, the effective concentration of an induction agent will be below a critical concentration such that the induction produces the desired effect essentially without undesirable effects. As used herein, the term “critical concentration” refers to a concentration of induction agent above which undesirable effects are produced. Undesirable effects that may be the result of a concentration exceeding the critical concentration include but are not limited to, e.g., off-target effects (off-target activation of signaling, off-target inhibition of signaling), reduction or loss of function (e.g., loss of desired activator function, loss of desired inhibitor function) reduction of cell viability, increase in cell mortality, lineage restriction towards an undesired cell type, differentiation into an undesired cell type, loss of expression of a particular desired marker, etc. Whether a particular induction agent will have a critical concentration and what the critical concentrations of those agents having a critical concentration are will depend on the agent and the specific conditions in which the agent is used.

In some instances, cells of the instant disclosure may be contacted with multiple induction agents and/or multiple induction compositions in order achieve a desired cell type of derivative thereof. In some instances, a particular induction composition will contain two or more induction agents such that a particular cell culture is simultaneously contacted with multiple induction agents. In some instances, a particular series of induction compositions may be used, one at a time, in generating a desired mesodermal cell type such that a particular cell culture is successively contacted with multiple induction agents.

The duration of contact of a particular induction composition with a particular cell type, in some instances, may be referred to as the “exposure time” and exposure times may range from a day to weeks or more, including but not limited to e.g., 1 day, 1.5 days, 2 days, 2.5 days, 3 days, 3.5 days, 4 days, 4.5 days, 5 days, 5.5 days, 6 days, 6.5 days, 7 days, 7.5 days, 8 days, 8.5 days, 9 days, 9.5 days, 10 days, 11 days, 12, days, 13, days, 14 days, 15, days, etc. As used herein, exposure times are, in some instances, referred as consisting essentially of, e.g., 24 hours, indicating that the exposure time may be longer or shorter than that specified including those exposure times that are longer or shorter but do not materially affect the basic outcome of the particular exposure. As such, in some instances where a particular exposure is more time sensitive such that under or over exposure, e.g., of more or less than 1 hour, materially affects the outcome of the exposure, a time period consisting essentially of, e.g., 24 hours, will be interpreted to refer to a time period ranging from about 23 hours to about 25 hours. In some other instances where a particular exposure is less time sensitive such that under or over exposure, e.g., of more than 12 hours, does not materially affect the outcome of the exposure, a time period consisting essentially of, e.g., 24 hours will mean a time period ranging from about 12 hours or less to about 36 hours or more. In some instances, depending on the context, an exposure period consisting essentially of 24 hours may refer to an exposure time of 22-26 hours, 21-27 hours, 20-28 hours, 19-29 hours, 18-30 hours, etc.

In some instances, time periods of exposure may be pre-determined such that cells are contacted with an induction composition according to a schedule set forth prior to the contacting. In some instances, the time period of exposure, whether pre-determined or otherwise, may be modulated according to some feature or characteristic of the cells and/or cell culture, including but not limited to, e.g., cell morphology, cell viability, cell appearance, cellular behaviors, cell number, culture confluence, marker expression, etc.

### Factors

Inhibitors of the TGF-beta pathway include but are not limited to, e.g., A-83-01 (3-(6-Methyl-2-pyridinyl)-N-phenyl-4-(4-quinolinyl)-1H-pyrazole-1-carbothioamide), D4476 (4-[4-(2,3-Dihydro-1,4-benzodioxin-6-yl)-5-(2-pyridinyl)-1H-imidazol-2-yl]benzamide), GW 788388 (4-[4-[3-(2-Pyridinyl)-1H-pyrazol-4-yl]-2-pyridinyl]-N-(tetrahydro-2H-pyran-4-yl)-benzamide), LY 364947 (4-[3-(2-Pyridinyl)-1H-pyrazol-4-yl]-quinoline), RepSox (2-(3-(6-Methylpyridine-2-yl)-1H-pyrazol-4-yl)-1,5-naphthyridine), SB431542 (4-[4-(1,3-benzodioxol-5-yl)-5-(2-pyridinyl)-1H-imidazol-2-yl]benzamide), SB-505124 (2-[4-(1,3-Benzodioxol-5-yl)-2-(1,1-dimethylethyl)-1H-imidazol-5-yl]-6-methyl-pyridine), SB 525334 (6-[2-(1,1-Dimethylethyl)-5-(6-methyl-2-pyridinyl)-1H-imidazol-4-yl]quinoxaline), SD208 (2-(5-Chloro-2-fluorophenyl)-4-[(4-pyridyl)amino]pteridine), ITD1 (4-[1,1′-Biphenyl]-4-yl-1,4,5,6,7,8-hexahydro-2,7,7-trimethyl-5-oxo-3-quinolinecarboxylic acid ethyl ester), DAN/Fc, antibodies to TGF-beta and TGF-beta receptors, TGF-beta inhibitory nucleic acids, and the like.

Activators of the WNT pathway include but are not limited to, e.g., CHIR99021 (6-[[2-[[4-(2,4-Dichlorophenyl)-5-(5-methyl-1H-imidazol-2-yl)-2-pyrimidinyl]amino]ethyl]amino]-3-pyridinecarbonitrile), WNT family ligands (e.g., including but not limited to Wnt-1, Wnt-2, Wnt-2b, Wnt-3a, Wnt-4, Wnt-5a, Wnt-5b, Wnt-6, Wnt-7a, Wnt-7a/b, Wnt-7b, Wnt-8a, Wnt-8b, Wnt-9a, Wnt-9b, Wnt-10a, Wnt-10b, Wnt-11, Wnt-16b, etc.), RSPO co-agonists (e.g., RSPO2), lithium chloride, TDZD8 (4-Benzyl-2-methyl-1,2,4-thiadiazolidine-3,5-dione), BIO-Acetoxime ((2′Z,3′E)-6-Bromoindirubin-3′-acetoxime), A1070722 (1-(7-Methoxyquinolin-4-yl)-3-[6-(trifluoromethyl)pyridin-2-yl]urea), HLY78 (4-Ethyl-5,6-Dihydro-5-methyl-[1,3]dioxolo[4,5-j]phenanthridine), CID 11210285 hydrochloride (2-Amino-4-(3,4-(methylenedioxy)benzylamino)-6-(3-methoxyphenyl)pyrimidine hydrochloride), WAY-316606, (hetero)arylpyrimidines, IQ1, QS11, SB-216763, DCA, and the like. In some instances, activation of the Wnt pathway may be achieved through repression of the a Wnt pathway inhibitor, e.g., including but not limited to the use of an inhibitory nucleic acid targeting an inhibitor of the Wnt pathway or an antibody or small molecule directed to a Wnt pathway inhibitor.

Inhibitors of the WNT pathway include but are not limited to, e.g., C59 (4-(2-Methyl-4-pyridinyl)-N-[4-(3-pyridinyl)phenyl]benzeneacetamide), DKK1, IWP-2 (N-(6-Methyl-2-benzothiazolyl)-2-[(3,4,6,7-tetrahydro-4-oxo-3-phenylthieno[3,2-d]pyrimidin-2-yl)thio]-acetamide), Ant1.4Br, Ant 1.4C1, Niclosamide, apicularen, bafilomycin, XAV939 (3,5,7,8-Tetrahydro-2-[4-(trifluoromethyl)phenyl]-4H-thiopyrano[4,3-d]pyrimidin-4-one), IWR-1 (4-(1,3,3a,4,7,7a-Hexahydro-1,3-dioxo-4,7-methano-2H-isoindol-2-yl)-N-8-quinolinyl-Benzamide), NSC668036 (N-[(1,1-Dimethylethoxy)carbonyl]-L-alanyl-(2S)-2-hydroxy-3-methylbutanoyl-L-Alanine-(1S)-1-carboxy-2-methylpropyl ester hydrate), 2,4-diamino-quinazoline, Quercetin, ICG-001 ((6S,9aS)-Hexahydro-6-[(4-hydroxyphenyl)methyl]-8-(1-naphthalenylmethyl)-4,7-dioxo-N-(phenylmethyl)-2H-pyrazino[1,2-a]pyrimidine-1(6H)-carboxamide), PKF115-584, BML-284 (2-Amino-4-[3,4-(methylenedioxy)benzylamino]-6-(3-methoxyphenyl)pyrimidine), FH-535, iCRT-14, JW-55, JW-67, antibodies to Wnts and Wnt receptors, Wnt inhibitory nucleic acids, and the like.

Activators of the FGF pathway and/or the MAPK/ERK pathway include but are not limited to, e.g., FGF family ligands (e.g., FGF1, FGF2, FGF-3, FGF-4, FGF-5, FGF-6, KGF/FGF-7, FGF-8, FGF-9, FGF-10, FGF-11, FGF-12, FGF-13, FGF-15, FGF-16, FGF-17, FGF-19, FGF-20, FGF-21, FGF-22, FGF-23, etc.), SUN 11602 (4-[[4-[[2-[(4-Amino-2,3,5,6-tetramethylphenyl)amino]acetyl]methylamino]-1-piperidinyl]methyl]benzamide), t-Butylhydroquinone, U-46619, C2 Ceramide, Lactosyl Ceramide, Angiotensin I1, Baicalin, and the like. In some instances, activation of the FGF pathway and/or the MAPK/ERK pathway may be achieved through repression of the a FGF pathway and/or the MAPK/ERK pathway inhibitor, e.g., including but not limited to the use of an inhibitory nucleic acid targeting an inhibitor of the FGF pathway and/or the MAPK/ERK pathway or an antibody or small molecule directed to a FGF pathway inhibitor and/or MAPK/ERK pathway inhibitor.

### Differentiation of Epicardial and Cardiac Fibroblast Cells

Cardiac progenitor cells are differentiated from human pluripotent cells by (i) culture in the presence of an effective concentration of a Wnt agonist, including without limitation CHIR99021 of from about 0.1 μM to about 100 μM, and may be from about 1 μM to about 10 μM for a period of from about 24 to about 72 hours, and may be not more than about 48 hours. The cells are then (ii) recovered in medium, e.g. RPMI+B27-insulin for a period of from about 12 to about 36 hours, and the period may be around 24 hours. Following recovery, the cells are (iii) cultured in the presence of an effective concentration of a Wnt inhibitor, including without limitation IWR-1 of from about 0.1 μM to about 100 μM, and may be from about 1 μM to about 10 μM for a period of from about 24 to about 72 hours, and may be about 48 hours. The cells are then (iv) recovered in medium, e.g. RPMI+B27-insulin for a period of from about 12 to about 36 hours, and the period may be around 24 hours.

The cardiac progenitor cells are plated in medium, e.g. advanced DMEM medium, for a period of from about 1 to about 4 days, e.g. 2 days, 3 days, etc. The cells are then (v) cultured in the presence of an effective concentration of a Wnt agonist, including without limitation CHIR99021 of from about 0.1 μM to about 100 μM, and may be from about 1 μM to about 10 μM; and retinoic acid, e.g. from about 0.1 μM to about 100 μM, and may be from about 1 μM to about 10 μM for a period of from about 24 to about 72 hours, and may be not more than about 48 hours to generate a population of human epicardial cells.

To generate cardiac fibroblasts, the epicardial cells are cultured in the presence of an effective concentration of an FGF agonist, e.g. FGF2; and a TGF-β inhibitor, e.g. SB431542; each at a concentration of from about 0.1 μM to about 100 μM, and may be from about 1 μM to about 50 μM, from about 5 μM to about 25 μM, and may be about 10 μM, for a period of from about 4, about 5, about 6, about 7, about 8, about 9, about 10 days or more. The period may be from about 5 to about 8 days, e.g. around 6 days.

### Screening Assays

Methods are provided for determining the activity of a candidate agent on a cardiac fibroblast, the method comprising contacting the candidate agent with one or a panel of cardiac fibroblasts differentiated from induced human pluripotent stem cells (iPS cells); and determining the effect of the agent on morphologic, genetic or functional parameters, e.g. indications of fibrosis, such as altered morphology, overproduction of collagen, and the like.

In screening assays for the small molecules, the effect of adding a candidate agent to cells in culture is tested with a panel of cells and cellular environments, where the cellular environment includes one or more of: electrical stimulation including alterations in ionicity, drug stimulation, and the like, and where panels of cells may vary in genotype, in prior exposure to an environment of interest, in the dose of agent that is provided, etc., where usually at least one control is included, for example a negative control and a positive control. Culture of cells is typically performed in a sterile environment, for example, at 37° C. in an incubator containing a humidified 92-95% air/5-8% CO₂ atmosphere. Cell culture may be carried out in nutrient mixtures containing undefined biological fluids such as fetal calf serum, or media which is fully defined and serum free. The effect of the altering of the environment is assessed by monitoring multiple output parameters, including morphological, functional and genetic changes.

In the screening assays for genetic agents, polynucleotides are added to one or more of the cells in a panel in order to alter the genetic composition of the cell. The output parameters are monitored to determine whether there is a change in phenotype. In this way, genetic sequences are identified that encode or affect expression of proteins in pathways of interest. The results can be entered into a data processor to provide a screening results dataset. Algorithms are used for the comparison and analysis of screening results obtained under different conditions.

Parameters are quantifiable components of cells, particularly components that can be accurately measured, desirably in a high throughput system. A parameter can also be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Variability is expected and a range of values for each of the set of test parameters will be obtained using standard statistical methods with a common statistical method used to provide single values.

Parameters of interest include detection of cytoplasmic, cell surface or secreted biomolecules, frequently biopolymers, e.g. polypeptides, polysaccharides, polynucleotides, lipids, etc. Cell surface and secreted molecules are a preferred parameter type as these mediate cell communication and cell effector responses and can be more readily assayed. In one embodiment, parameters include specific epitopes. Epitopes are frequently identified using specific monoclonal antibodies or receptor probes. In some cases the molecular entities comprising the epitope are from two or more substances and comprise a defined structure; examples include combinatorially determined epitopes associated with heterodimeric integrins. A parameter may be detection of a specifically modified protein or oligosaccharide. A parameter may be defined by a specific monoclonal antibody or a ligand or receptor binding determinant.

Candidate agents of interest are biologically active agents that encompass numerous chemical classes, primarily organic molecules, which may include organometallic molecules, inorganic molecules, genetic sequences, etc. An important aspect of the invention is to evaluate candidate drugs, select therapeutic antibodies and protein-based therapeutics, with preferred biological response functions. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, anti-inflammatory agents, hormones or hormone antagonists, ion channel modifiers, and neuroactive agents. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Cardiovascular Drugs; Vitamins, Dermatology; and Toxicology, all incorporated herein by reference.

Test compounds include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

The term samples also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1:1 to 1 ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

As used herein, the term “genetic agent” refers to polynucleotides and analogs thereof, which agents are tested in the screening assays of the invention by addition of the genetic agent to a cell. The introduction of the genetic agent results in an alteration of the total genetic composition of the cell. Genetic agents such as DNA can result in an experimentally introduced change in the genome of a cell, generally through the integration of the sequence into a chromosome. Genetic changes can also be transient, where the exogenous sequence is not integrated but is maintained as an episomal agents. Genetic agents, such as antisense oligonucleotides, can also affect the expression of proteins without changing the cell's genotype, by interfering with the transcription or translation of mRNA. The effect of a genetic agent is to increase or decrease expression of one or more gene products in the cell.

Introduction of an expression vector encoding a polypeptide can be used to express the encoded product in cells lacking the sequence, or to over-express the product. Various promoters can be used that are constitutive or subject to external regulation, where in the latter situation, one can turn on or off the transcription of a gene. These coding sequences may include full-length cDNA or genomic clones, fragments derived therefrom, or chimeras that combine a naturally occurring sequence with functional or structural domains of other coding sequences. Alternatively, the introduced sequence may encode an anti-sense sequence; be an anti-sense oligonucleotide; RNAi, encode a dominant negative mutation, or dominant or constitutively active mutations of native sequences; altered regulatory sequences, etc.

Antisense and RNAi oligonucleotides can be chemically synthesized by methods known in the art. Preferred oligonucleotides are chemically modified from the native phosphodiester structure, in order to increase their intracellular stability and binding affinity. A number of such modifications have been described in the literature, which after the chemistry of the backbone, sugars or heterocyclic bases. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity, e.g. morpholino oligonucleotide analogs. The □-anomer of deoxyribose may be used, where the base is inverted with respect to the natural □-anomer. The 2′-OH of the ribose sugar may be altered to form 2′-O-methyl or 2′-O-allyl sugars, which provides resistance to degradation without comprising affinity.

Agents are screened for biological activity by adding the agent to at least one and usually a plurality of cells, in one or in a plurality of environmental conditions, e.g. following stimulation with a β-adrenergic agonist, following electric or mechanical stimulation, etc. The change in parameter readout in response to the agent is measured, desirably normalized, and the resulting screening results may then be evaluated by comparison to reference screening results, e.g. with cells having other mutations of interest, normal cardiac fibroblasts, cardiac fibroblasts derived from other family members, and the like. The reference screening results may include readouts in the presence and absence of different environmental changes, screening results obtained with other agents, which may or may not include known drugs, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Various methods can be utilized for quantifying the presence of selected parameters, in addition to the functional parameters described above. For measuring the amount of a molecule that is present, a convenient method is to label a molecule with a detectable moiety, which may be fluorescent, luminescent, radioactive, enzymatically active, etc., particularly a molecule specific for binding to the parameter with high affinity Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein chimeras inside cells (for a review see Jones et al. (1999) Trends Biotechnol. 17(12):477-81). Thus, antibodies can be genetically modified to provide a fluorescent dye as part of their structure

Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. These techniques utilize specific antibodies as reporter molecules, which are particularly useful due to their high degree of specificity for attaching to a single molecular target. U.S. Pat. No. 4,568,649 describes ligand detection systems, which employ scintillation counting. These techniques are particularly useful for protein or modified protein parameters or epitopes, or carbohydrate determinants. Cell readouts for proteins and other cell determinants can be obtained using fluorescent or otherwise tagged reporter molecules. Cell based ELISA or related non-enzymatic or fluorescence-based methods enable measurement of cell surface parameters and secreted parameters. Capture ELISA and related non-enzymatic methods usually employ two specific antibodies or reporter molecules and are useful for measuring parameters in solution. Flow cytometry methods are useful for measuring cell surface and intracellular parameters, as well as shape change and granularity and for analyses of beads used as antibody- or probe-linked reagents. Readouts from such assays may be the mean fluorescence associated with individual fluorescent antibody-detected cell surface molecules or cytokines, or the average fluorescence intensity, the median fluorescence intensity, the variance in fluorescence intensity, or some relationship among these.

Both single cell multiparameter and multicell multiparameter multiplex assays, where input cell types are identified and parameters are read by quantitative imaging and fluorescence and confocal microscopy are used in the art, see Confocal Microscopy Methods and Protocols (Methods in Molecular Biology Vol. 122.) Paddock, Ed., Humana Press, 1998. These methods are described in U.S. Pat. No. 5,989,833 issued Nov. 23, 1999.

The quantitation of nucleic acids, especially messenger RNAs, is also of interest as a parameter. These can be measured by hybridization techniques that depend on the sequence of nucleic acid nucleotides. Techniques include polymerase chain reaction methods as well as gene array techniques. See Current Protocols in Molecular Biology, Ausubel et al., eds, John Wiley & Sons, New York, N.Y., 2000: Freeman et al. (1999) Biotechniques 26(1):112-225; Kawamoto et al. (1999) Genome Res 9(12):1305-12; and Chen et al. (1998) Genomics 51(3):313-24, for examples.

The comparison of a screening results obtained from a test compound, and a reference screening results(s) is accomplished by the use of suitable deduction protocols, AI systems, statistical comparisons, etc. Preferably, the screening results is compared with a database of reference screening results. A database of reference screening results can be compiled. These databases may include reference results from panels that include known agents or combinations of agents, as well as references from the analysis of cells treated under environmental conditions in which single or multiple environmental conditions or parameters are removed or specifically altered. Reference results may also be generated from panels containing cells with genetic constructs that selectively target or modulate specific cellular pathways.

The readout may be a mean, average, median or the variance or other statistically or mathematically derived value associated with the measurement. The parameter readout information may be further refined by direct comparison with the corresponding reference readout. The absolute values obtained for each parameter under identical conditions will display a variability that is inherent in live biological systems and also reflects individual cellular variability as well as the variability inherent between individuals.

For convenience, the systems of the subject invention may be provided in kits. The kits could include the cells to be used, which may be frozen, refrigerated or treated in some other manner to maintain viability, reagents for measuring the parameters, and software for preparing the screening results. The software will receive the results and perform analysis and can include reference data. The software can also normalize the results with the results from a control culture. The composition may optionally be packaged in a suitable container with written instructions for a desired purpose, such as screening methods, and the like.

For further elaboration of general techniques useful in the practice of this invention, the practitioner can refer to standard textbooks and reviews in cell biology, tissue culture, embryology, and cardiophysiology. With respect to tissue culture and embryonic stem cells, the reader may wish to refer to Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998). With respect to the culture of heart cells, standard references include The Heart Cell in Culture (A. Pinson ed., CRC Press 1987), Isolated Adult Cardiomyocytes (Vols. I & II, Piper & Isenberg eds, CRC Press 1989), Heart Development (Harvey & Rosenthal, Academic Press 1998).

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

Each publication cited in this specification is hereby incorporated by reference in its entirety for all purposes.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXAMPLES

### Example 1

Generation of Quiescent Cardiac Fibroblasts from Human Induced Pluripotent Stem Cells for in vitro Modeling of Cardiac Fibrosis

Activated fibroblasts are the major cell type that secrete excessive extracellular matrix in response to injury, contributing to pathological fibrosis and leading to organ failure. Effective anti-fibrotic therapeutic solutions, however, are not available due to the poorly defined characteristics and unavailability of tissue-specific fibroblasts. Recent advances in single-cell RNA-sequencing (scRNA-seq) fill such gaps of knowledge by enabling delineation of the developmental trajectories and identification of regulatory pathways of tissue-specific fibroblasts among different organs. By analyzing the single-cell transcriptome profiles of fibroblasts from selected mouse tissues, we identified distinct tissue-specific signature genes, including transcription factors that define the identities of fibroblasts in the heart, lungs, trachea, and bladder. We also determined that CFs in large are of the epicardial lineage. We thus developed a robust chemically-defined protocol that generates CFs from human iPSCs. Functional studies confirmed that iPSC-derived CFs preserved a quiescent phenotype and highly resembled primary CFs at the transcriptional, cellular, and functional levels. We demonstrated that this cell-based platform is sensitive to both pro- and anti-fibrosis drugs. Finally, we showed that crosstalk between cardiomyocytes and CFs via the atrial/brain natriuretic peptide-natriuretic peptide receptor pathway is implicated in suppressing fibrogenesis.

This study uncovers unique gene signatures that define tissue-specific identities of fibroblasts. The bona fide quiescent CFs derived from human iPSCs can serve as a faithful in vitro platform to better understand the underlying mechanisms of cardiac fibrosis and to screen antifibrotic drugs.

### Abbreviations and Acronyms

ANP atrial natriuretic peptide
BNP brain natriuretic peptide
CF cardiac fibroblast
CM cardiomyocyte
CPC cardiac progenitor cell
ECM extracellular matrix
EPC epicardial cell
FGF2 fibroblast growth factor 2
GO gene ontology
iPSC induced pluripotent stem cell
NPPA/NPPB natriuretic peptide type A/B
NPR1 natriuretic peptide receptor 1
α-SMA α-smooth muscle actin

### SB SB431542

scRNA-seq single-cell RNA-sequencing
TF transcription factors
TGF-β transforming growth factor-β
t-SNE t-distributed stochastic neighbor embedding

By analyzing the recently reported Tabula Muris, a mouse atlas of single-cell transcriptome from >100,000 cells across 20 tissues, we report here that fibroblasts carry tissue-specific transcriptomic signatures that are conserved in humans. We found that CFs in large are of the epicardial lineage based on specifically expressed epicardial transcription factors (TFs) in these cells. We used these insights to develop a robust protocol for generating human iPSC-CFs that closely resemble quiescent human primary CFs at the transcriptional, cellular, and functional levels. We further showed that human iPSC-CFs can serve as a powerful in vitro platform to better understand the mechanisms of cardiac fibrosis and to screen anti- and pro-fibrotic drugs.

Collectively, our study used comprehensive scRNA-seq data to define tissue-specific genes and pathways in fibroblasts. Importantly, the human iPSC-based induction protocol we developed can be adapted to generate patient-specific CFs, which may pave the way to reveal the complexity of cardiac fibrosis and thereby offer a new direction for precision medicine.

### Results

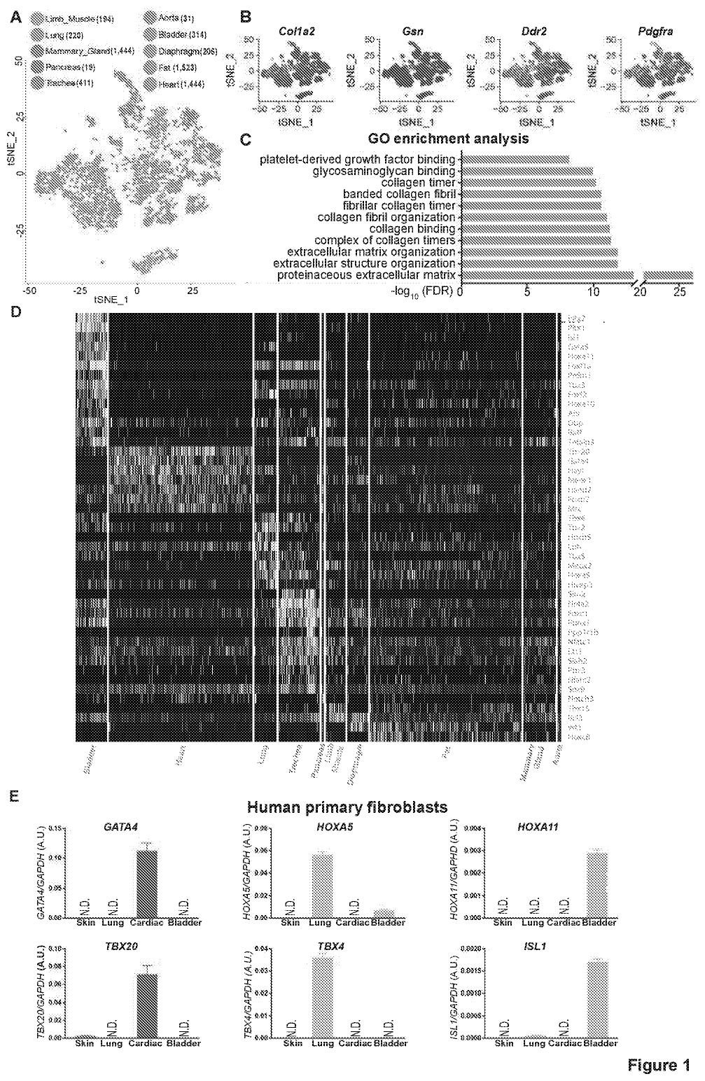

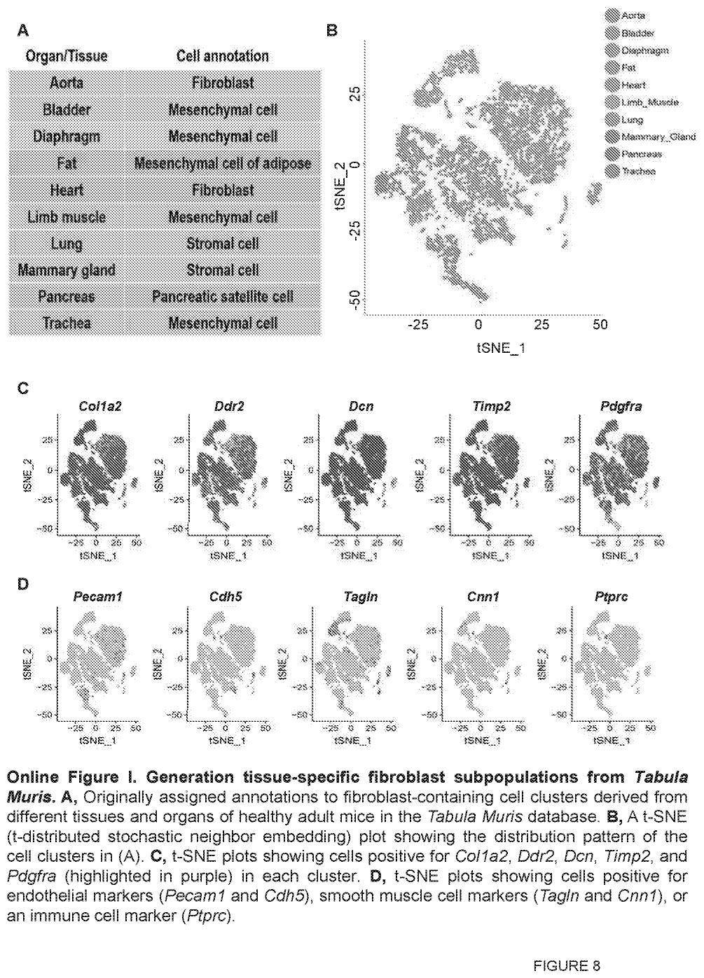

Mouse Single-Cell Transcriptome Reveals Tissue-Specific Gene Markers for Fibroblasts are Conserved in Humans. Because specific genes that can distinguish fibroblasts from other cell types are not yet available, we first subtracted the transcriptome profiles of fibroblast-containing cell clusters in 10 selected tissues from the Tabula Muris database (Online FIG. 1A). 5 We then mapped these fibroblast containing cell clusters on a t-distributed stochastic neighbor embedding (t-SNE) plot, and refined the fibroblast population in each tissue by selecting cells that are positive for genes (Col1a2, Ddr2, Dcn, Timp2, and Pdgfra) highly expressed in fibroblasts, and by excluding those expressing any of the gene markers for endothelial cells (Pecam1 and Cdh5), smooth muscle cells (Tagin and Cnn1), or immune cells (Ptprc) (FIG. 8D).

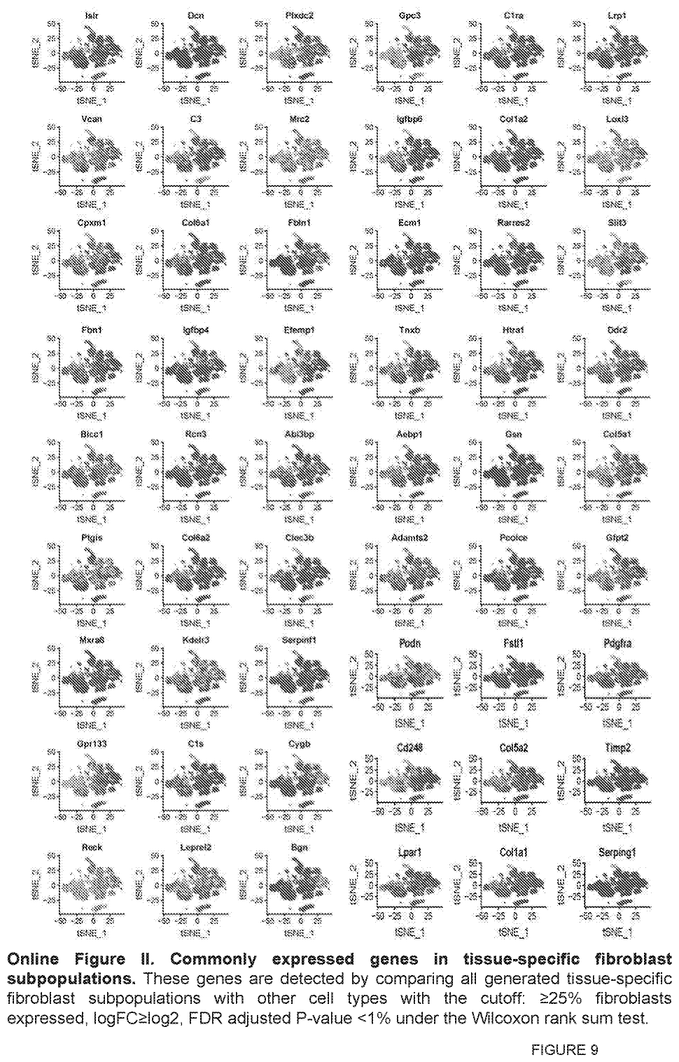

Ultimately, we identified a total of 4,685 out of 44,949 cells as fibroblasts in the Tabula Muris dataset, and mapped them on a new t-SNE plot (FIG. 1A). As expected, these cells express common fibroblast marker genes (FIG. 1B and FIG. 9). Moreover, gene ontology (GO) enrichment analysis revealed that these genes are enriched in fibroblast-related signaling pathways, such as proteinaceous extracellular matrix, extracellular structure organization, and platelet derived growth factor binding (FIG. 1C).

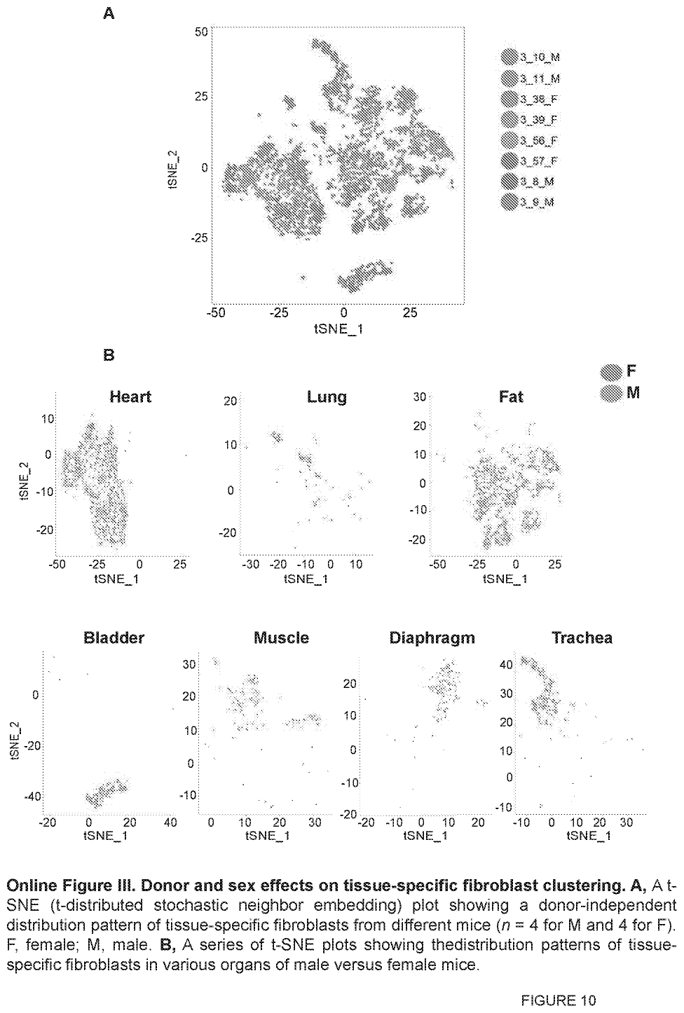

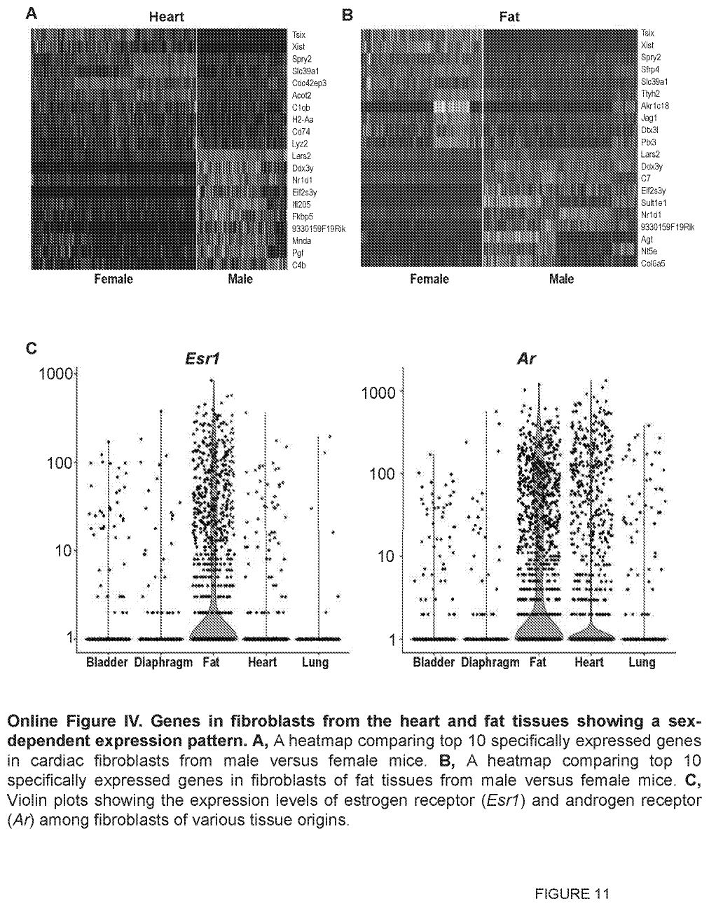

Next, we examined which factors are responsible for separating fibroblasts into different clusters (FIG. 1A). After pooling fibroblasts from each mouse and mapping them on a new t-SNE plot, we did not observe any donor effects that contributed to the specificity of fibroblast clustering in different tissues (FIG. 10A). However, fibroblasts in the heart and fat tissues were separated by sex (FIG. 10B). Further analysis revealed that the sex-polarized gene expression patterns (FIGS. 11A and 11B) might be associated with higher expression levels of sex hormonal receptors in the heart and fat tissues compared to other organs (FIG. 11C).

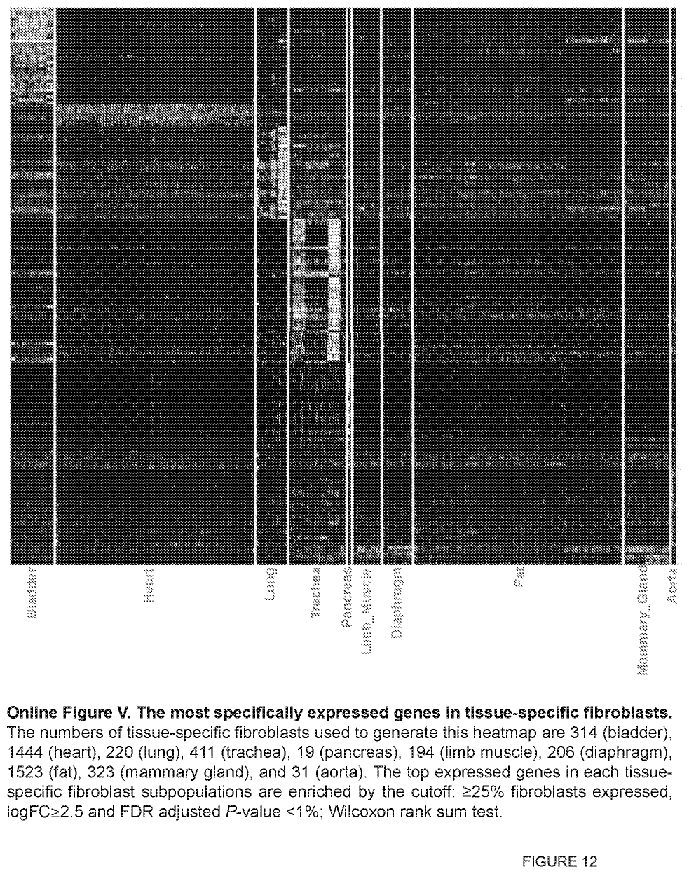

We then compared the transcriptome profiles between tissue-specific fibroblasts, and observed that fibroblasts derived from the heart, lungs, bladder, and trachea demonstrated distinct gene signatures (FIG. 12). Moreover, we observed that fibroblasts in these four organs also exhibit a tissue-specific distribution pattern of TFs (FIG. 1D). TFs have been suggested to play a critical role in defining cell identities. 6, 7 Tbx20, Gata4, and Hand2 are specifically enriched in CFs, whereas Tbx2, Tbx4, and Hoxa5 are highly expressed in lung fibroblasts. Fibroblasts from the aorta, diaphragm, fat, mammary gland, and limb muscle did not show an apparent tissue-specific pattern for TFs (FIG. 1D). Intriguingly, human primary fibroblasts isolated from the heart, lungs, and bladder also specifically expressed GATA4/TBX20, HOXA5/TBX4, and HOXA11/ISL1, respectively (FIG. 1E). Together, the single-cell transcriptome suggests that the identities of fibroblasts in different mouse tissues are distinguished and probably determined by a handful of conserved tissue-specific signature genes.

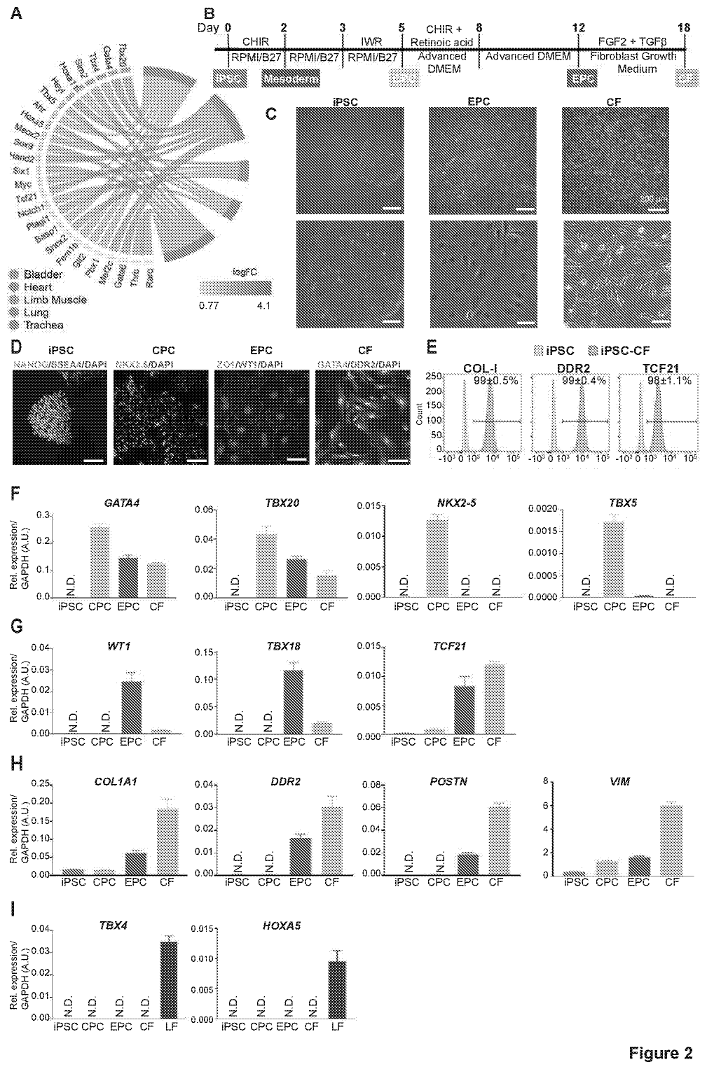

Generation of Quiescent Human iPSC-CFs. By performing the GO pathway enrichment analysis on TFs, we observed that some of these genes are enriched in a tissue-specific development pathway (FIG. 2A). For example, we identified that Gata4, Gata6, Tbx20, Heyl, Hand2, Mef2c, and Tcf21 are enriched in the cardiac development pathway (FIG. 2A). Because GATA4 and TBX20 are highly expressed in cardiac mesoderm and TCF21 is a marker for epicardium, we hypothesized that CFs could be generated from human iPSCs based on this developmental program. Accordingly, we modified an established protocol designed to generate human iPSC-derived epicardial cells (EPCs) by continuing to differentiate the intermediate cells in a commercial fibroblast growth medium in the presence of fibroblast growth factor (FGF2) and transforming growth factor beta (TGF-β) inhibitor SB431542 (SB) (FIG. 2B). After six days of extended culture (i.e., day 18 of differentiation), the cells showed a typical fibroblast morphology (FIG. 2C), and expressed markers (GATA4 and DDR2) that are highly expressed in primary CFs (FIG. 2D). Notably, flow cytometry analysis revealed that >90% of the cells were positive for COL-I, DDR2, and TCF21 compared with undifferentiated iPSCs (FIG. 2E).

We then examined the dynamic changes of the transcriptional profiles of cardiac and fibroblast-specific genes at various stages of differentiation. We observed that GATA4 and TBX20, two early cardiac-specific developmental marker genes, showed relative higher expression levels in iPSC-derived cardiac progenitor cells (CPCs) than in iPSC-EPCs and iPSC-CFs (FIG. 2F). By contrast, NKX2-5 and TBX5 were only transiently expressed in iPSC-CPCs. Epicardial markers WT1 and TBX18 became highly expressed in iPSC-EPCs but decreased in iPSC-CFs (FIG. 2G). It has been reported that TCF21-expressing EPCs are initially multipotent, and eventually become committed to the fibroblast lineage with the upregulation of TCF21.12 Consistently, TCF21 was found to be substantially increased in iPSC-EPCs and became more highly expressed in iPSC-CFs (FIG. 2G). As expected, the expression levels of typical fibroblast markers (COL1A1, DDR2, POSTN, and VIM) were gradually increased during differentiation (FIG. 2H), whereas lung specific development-related genes such as TBX4 and HOXA5 were not detectable at any stage of differentiation (FIG. 2I). Taken together, we confirm that iPSC-CFs obtained through the differentiation protocol used in this study express both cardiac and fibroblast-specific markers.

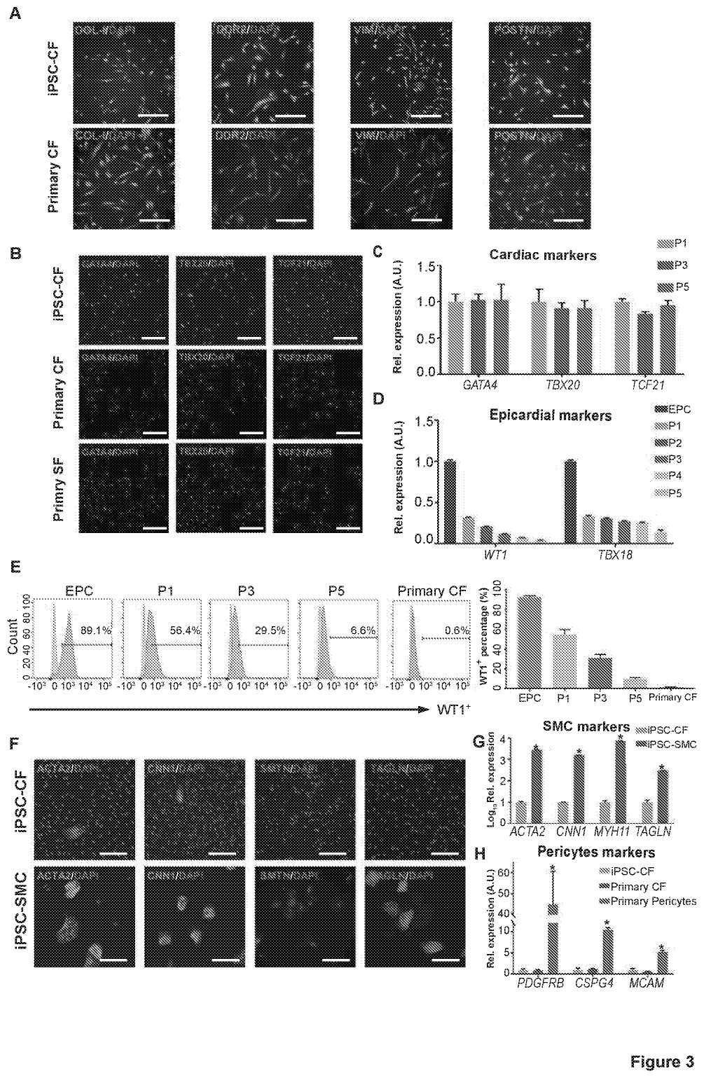

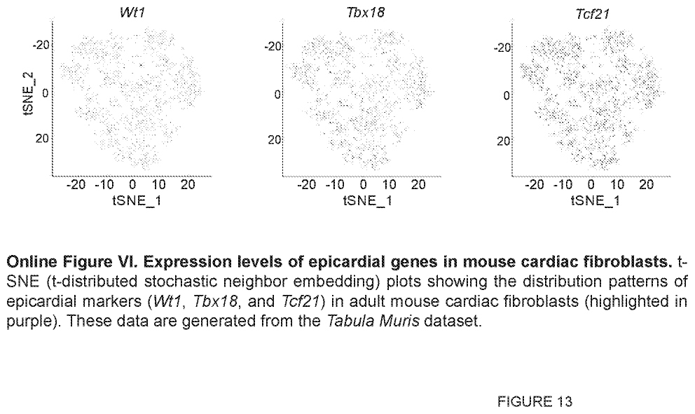

Human iPSC-CFs Preserve Their Cell Identity Profile During Passaging. In order to verify whether iPSC-CFs are bona fide cardiac fibroblasts, we next compared their transcriptomes and proteomes with primary cultures. Intriguingly, human iPSC-CFs expressed similar protein levels of typical fibroblast markers (COL-I, DDR2, VIM, and POSTN) and cardiac markers (GATA4, TBX20, and TCF21) as primary fibroblasts (FIGS. 3A and 3B). We also found that the human iPSC-CFs could be expanded for at least five passages without losing their cardiac fibroblast identity (FIG. 3C). As expected, the expression levels of TBX18 were persistently much lower in iPSC-CFs during serial passaging than in iPSC-EPCs (FIGS. 2G and 3D). While WT1 is hardly detectable in adult primary CFs, it decreased dramatically to less than 10% in human iPSC-CFs at passage 5 (FIG. 3D, 3E, and FIG. 13). Taken together, these data indicate that iPSC-CFs show a high similarity to primary CFs in terms of cell identity profiles.

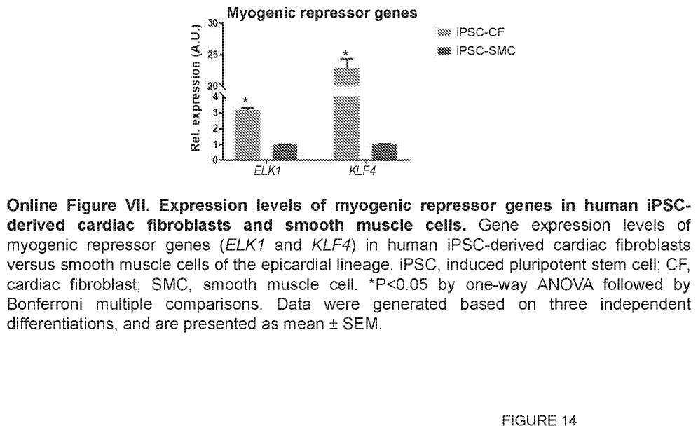

Because cardiac smooth muscle cells and pericytes are also derived from the epicardial lineage we blocked these potential differentiation trajectories by adding SB431542 (SB), a TGF-β inhibitor, during CF differentiation (FIG. 2B). As a result, human iPSC-CFs expressed negligible levels of smooth muscle marker genes (ACTA2, CNN1, TAGLN, SMTN, and MYH11) at both transcriptional and protein levels (FIGS. 3F and 3G). We also observed that human iPSC CFs expressed higher levels of myogenic repressor genes ELK1 and KLF415, 16 than derived smooth muscle cells of the epicardial lineage (FIG. 14). Moreover, human iPSC-CFs expressed significantly lower levels of pericyte markers (PDGFRB, CSPG4, and MCAM) compared to primary pericytes (FIG. 3H). Therefore, the human iPSC-CFs generated in this study are highly pure cultures and can preserve cardiac fibroblast identity during serial passaging.

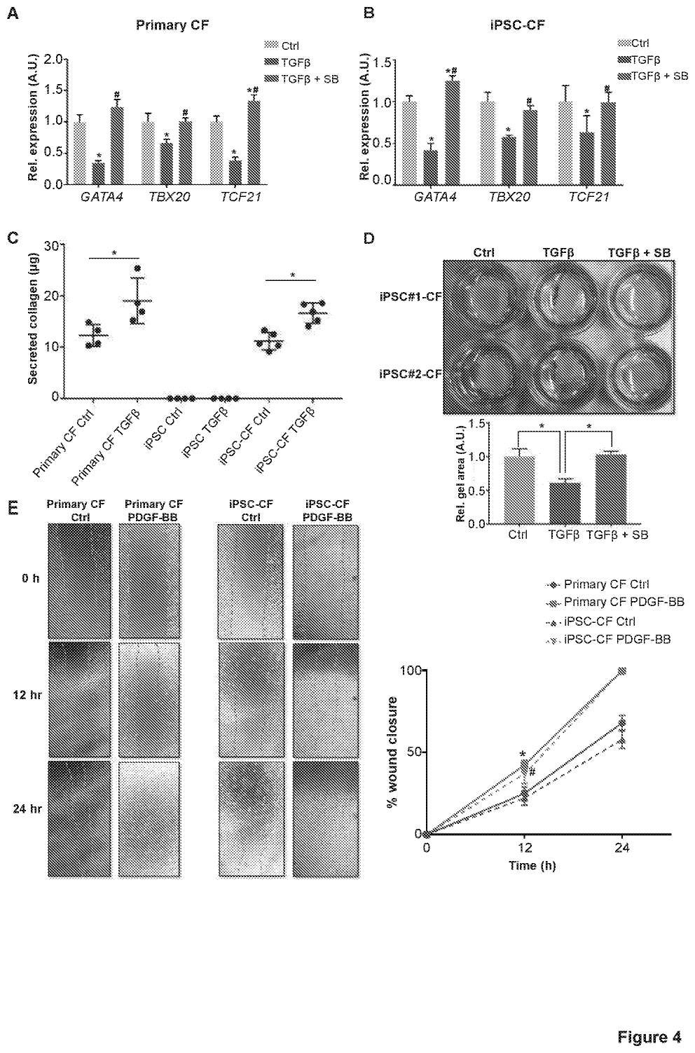

Human iPSC-CFs Closely Resemble Primary CFs. Next, we conducted a series of functional studies comparing human primary and iPSC-CFs. Under pathological conditions, upregulated TGF-β is one of the potent effectors that can stimulate fibroblast-to-myofibroblast transdifferentiation and thereby contribute to tissue fibrosis. Interestingly, we observed that the expression levels of cardiac-specific genes (GATA4, TBX20, and TCF21) were significantly downregulated in TGF-β-treated primary CFs as compared with vehicle-treated cells (FIG. 4A). A similar phenotype was also observed in human iPSC-CFs after TGF-β treatment (FIG. 4B). Because a previous study reported that fibroblast-restricted depletion of Tbx20 is associated with thicker scar tissues in infarcted mouse hearts, the downregulation of these cardiac-specific TFs may be involved in promoting CF transdifferentiation and cardiac fibrosis. However, the precise role of cardiac-specific TFs in cardiac fibrosis warrants further investigation.

Aberrant collagen synthesis contributes to tissue fibrosis. Using a Sirius red-based colorimetric assay kit, we observed that human iPSC-CFs and primary CFs secreted comparable basal levels of collagen into culture media, which were not detectable in human iPSCs (FIG. 4C). A similar rise in collagen content was detected in both groups of CFs upon TGF-β treatment (FIG. 4C). Increased contractile activity is also a specific measure of fibroblast transdifferentiation. We seeded human iPSC-CFs on collagen membranes, and observed a stronger collagen gel contraction effect in the TGF-β treated group than the vehicle group, whereas this effect was completely abrogated by SB (FIG. 4D). Human iPSC-CFs and primary CFs responded similarly to platelet derived growth factor-BB-induced cell migration (FIG. 4E). It has been shown that cardiac myofibroblasts could lead to myosin heavy chain 7 re-expression during cardiac fibrosis in vivo.

Interestingly, this phenotype was successfully recapitulated by co-culturing human iPSC-derived cardiomyocytes (iPSC-CMs) with iPSC-CFs in the presence of TGF-β (Figure VIII). Taken together, these functional studies confirm that the human iPSC-CFs generated in this study are bona fide cardiac fibroblasts, which resemble the biological and physiological properties of their primary counterparts.

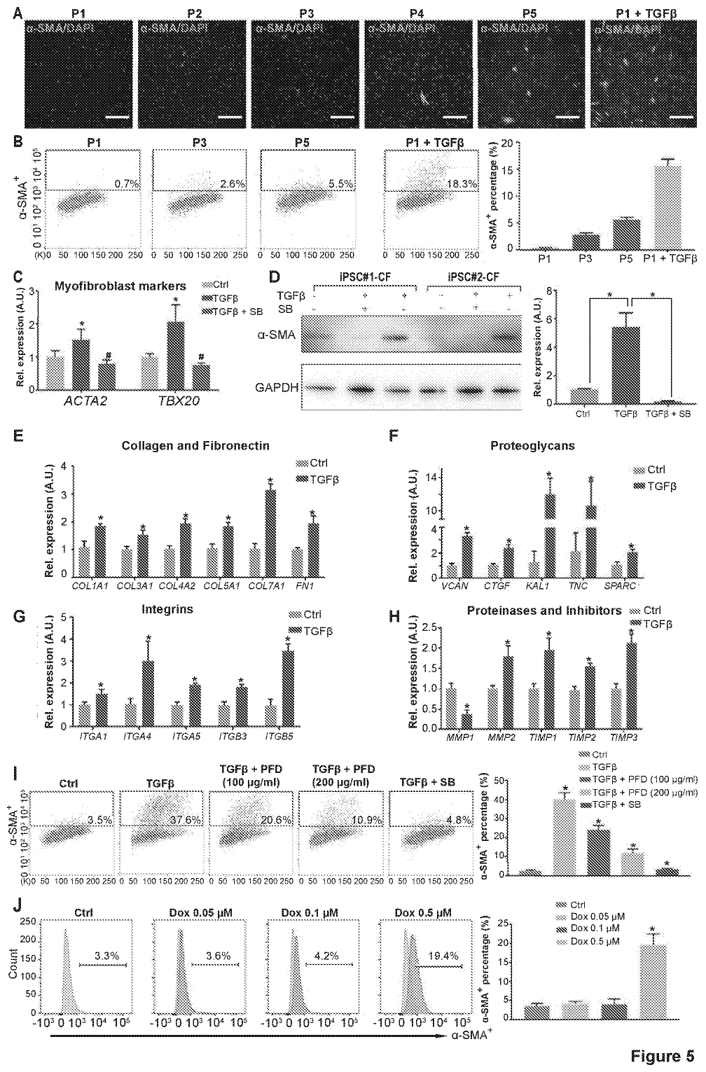

Quiescent Human iPSC-CFs Provide an in vitro Platform to Model Fibrosis. Previous studies have shown that primary fibroblasts can undergo spontaneous transdifferentiation into myofibroblasts during in vitro culturing. We hypothesized that inhibiting the TGF-β pathway during iPSC-CF differentiation may preserve their quiescent phenotype and suppress transdifferentiation. Using the α-smooth muscle actin (α-SMA) level as an indicator for the extent of fibroblast activation, we observed that the generated human iPSC-CFs were quiescent cells when the TGF-β pathway was inhibited (FIGS. 5A and 5B). Notably, most of these cells remained quiescent after several passages, and only about 5% of iPSC-CFs at passage 5 were positive for α-SMA (FIGS. 5A and 5B). Therefore, we used iPSC-CFs between passages 1-4 as a reliable cell model for the subsequent fibrosis studies in vitro.

We first confirmed that the expression levels of ACTA2 and POSTN were significantly increased in human iPSC-CFs upon TGF-β treatment as compared with the vehicle treatment (FIG. 5C). As expected, the addition of SB effectively inhibited the upregulation of these two myofibroblast markers (FIG. 5C). The α-SMA protein levels were also significantly increased in CFs derived from different human iPSC lines after TGF-β treatment, which were abolished by SB (FIG. 5D). Because the transdifferentiation from fibroblasts to myofibroblasts is strongly associated with the dynamic changes of ECM genes, we next performed a comprehensive ECM transcriptomic analysis on human iPSC-CFs after treatment with vehicle or TGF-β. We observed that the expression levels of ECM genes such as COL1A1, COL3A1, COL4A2, and FN1 were significantly increased by TGF-β (FIG. 5E). Similarly, several proteoglycans (VCAN, CTGF, KAL1, TNC, and SPARC) and cell-matrix focal adhesion molecules (ITGA4, ITGA5, and ITGB5) also showed upregulated expression in the presence of TGF-β (FIGS. 5F and 5G). Matrix metalloproteinases (MMPs) and their endogenous inhibitors (TIMPs) regulate the dynamic turnover of ECM. 21 We observed an upregulation of TIMP1, TIMP2, and TIMP3 concurrently with downregulation of MMP1 by TGF-β (FIG. 5H). Because MMP2 has been shown to be involved in activating TIMP2,22 it is not surprising that we observed an upregulation of MMP2 in iPSC-CFs after TGF-β treatment (FIG. 5H).

Next, we tested if the FDA-approved anti-idiopathic lung fibrosis drug pirfenidone could suppress TGF-β-induced transdifferentiation of human iPSC-CFs. Pirfenidone showed a dose dependent inhibitory effect on TGF-β-induced upregulation of α-SMA (FIG. 5I). Doxorubicin, a widely used chemotherapy drug, has been shown to cause several cardiac side effects, including cardiac fibrosis. Using human iPSC-CFs, we recapitulated doxorubicin-induced pro-fibrosis phenotype in vitro, which also showed a dose-dependent pattern (FIG. 5J). Together, these results show that the human iPSC-CFs generated in this study preserve a quiescent phenotype after differentiation and during early passages, making them sensitive to pro-fibrogenic stimuli. Therefore, these cells can serve as a faithful in vitro platform to screen drugs that can promote or suppress fibrosis.

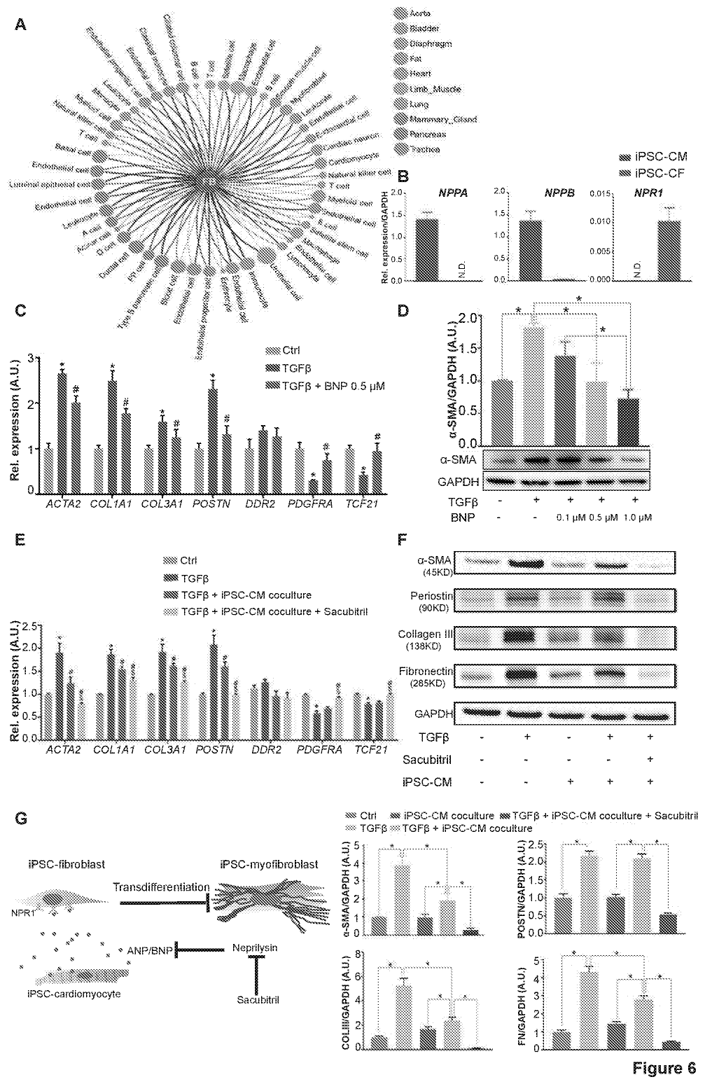

Crosstalk Between Human iPSC-CMs and iPSC-CFs Through the ANP/BNP-NPR1 Signaling Pathway Represents a Novel Strategy for Anti-Fibrotic Therapy. Cardiac fibroblasts are a key component of the heart that play a critical role in cardiac remodeling through dynamic cell-cell interactions. By carrying out complementary receptor-ligand interaction analysis (FIG. 6A), we observed that CFs are capable of interacting with a broad spectrum of cell types such as CMs, endothelial cells, smooth muscle cells, and immune cells through different signaling pathways. For example, we identified a novel interaction between ANP/BNP, the commonly used hypertrophic markers in CMs, and their cognate receptor NPR1 in CFs. We further verified that natriuretic peptide type A/B (NPPA/NPPB) and NPR1 are expressed in human iPSC-CMs and iPSC-CFs, respectively (FIG. 6B).

To understand whether the interaction through the ANP/BNP-NPR1 plays a role in cardiac fibrosis, we treated human iPSC-CFs with BNP in the presence of TGF-β. Interestingly, CFs expressed significantly lower mRNA levels of myofibroblast markers and ECM genes (ACTA2, POSTN, COL1A1, and COL3A1), and higher levels of CF markers (PDGFRA and TCF21) with BNP treatment (FIG. 6C). α-SMA protein levels were also significantly suppressed with escalating concentrations of BNP in a dose-dependent manner (FIG. 6D). We then co-cultured human iPSC-CFs with iPSC-CMs in the presence of TGF-β, and observed a similar expression pattern of these genes in co-culture as was seen with BNP treatment (FIG. 6E). The protein levels of myofibroblast markers and ECM proteins (α-SMA, collagen III, and fibronectin) in human iPSC-CFs were also significantly lower in the co-cultured than non-co-cultured condition (FIG. 6F).

ANP and BNP are known to be upregulated in cardiac hypertrophy and fibrosis. However, evidence shows that ANP and BNP have relatively short half-lives (3 minutes and 20 minutes, respectively) due to the ubiquitous presence of their degrading enzyme, neprilysin, in the circulatory system. As such, ANP/BNP may not be able to exert a long-lasting anti-fibrotic effect through the interaction with their receptor NPR1 expressed on CFs. Sacubitril, a neprilysin inhibitor, has been shown to ameliorate heart failure when used together with valsartan. Therefore, we further examined whether the addition of sacubitril to the co-culture system would further suppress CF transdifferentiation. Indeed, we observed that sacubitril further decreased the mRNA and protein levels of myofibroblast markers and ECM genes in the co-cultured condition (FIGS. 6E and 6F). In particular, the myofibroblast marker periostin was significantly decreased only when sacubitril was added to the co-culture system. Together, these data show that targeting the ANP/BNP-NPR1 signaling pathway can be a strategy for anti-cardiac fibrosis therapy (FIG. 6G).

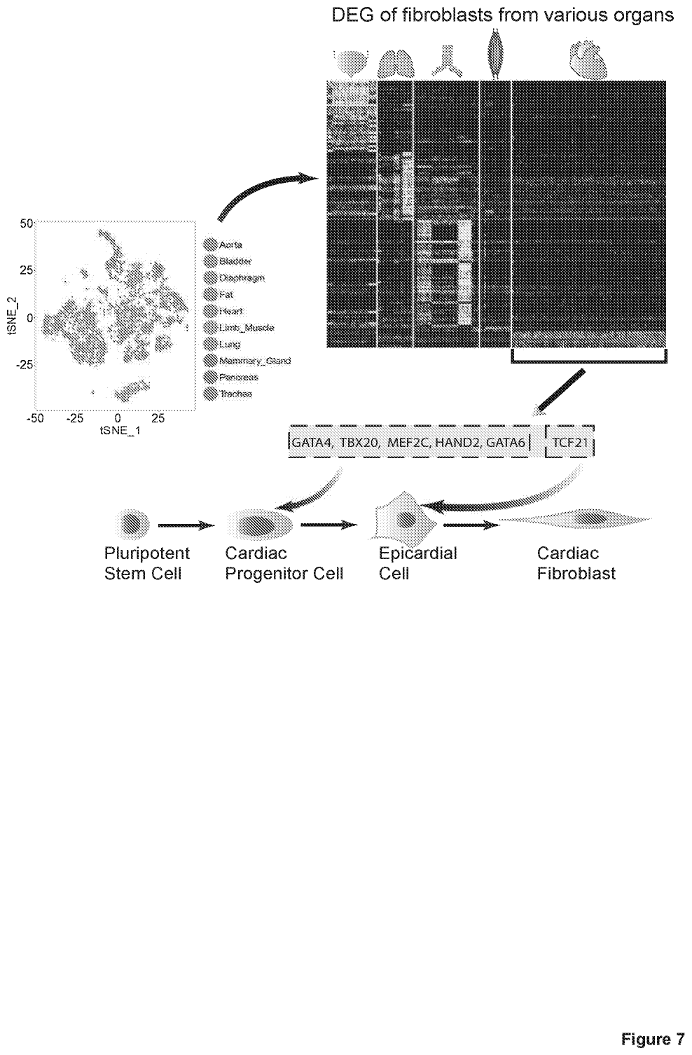

It is increasingly recognized that fibroblasts in different tissues are heterogeneous as evident by their distinct gene expression patterns. However, the inability to characterize the full transcriptomic picture of fibroblasts in a tissue-specific manner has impeded the development of mechanism-oriented, organ-specific anti-fibrotic therapies. In this study, by performing an in depth analysis of mouse single-cell transcriptomics data in combination with several assays, we identified a group of tissue-specific signature genes that are conserved in mouse and human primary fibroblasts. After identifying that CFs in large are of the epicardial lineage, we successfully developed a robust protocol to generate quiescent bona fide CFs from human iPSCs that could recapitulate most of the fundamental biological features of primary CFs (FIG. 7). We further demonstrated that human iPSC-CFs could be used as a faithful in vitro cell model to test drugs with pro- or anti-fibrosis potentials. Moreover, we identified that crosstalk between iPSC CMs and iPSC-CFs through the ANP/BNP-NPR1 pathway can represent a novel route for anticardiac fibrosis therapy. Our study also highlights the value of scRNA-seq in developing new candidate therapeutic strategies for treating tissue-specific fibrosis.

Accumulating evidence from animal studies suggests that cell lineage specification is governed by a handful of tissue-specific TFs. In this study, we observed that fibroblasts express high levels of tissue-specific, development-related TFs, raising the possibility that these genes may help define fibroblast tissue identity. Animal studies support this idea. Deficiency of Gata4 leads to failure to initiate proepicardium formation during cardiogenesis, and ablation of Tcf21 blocks the differentiation of CFs from proepicardial cells by suppressing epithelial-mesenchymal transition. In line with these in vivo findings, we observed a significant rise in GATA4 and TCF21 expression in human iPSC-CPCs and iPSC-EPCs, respectively. Collectively, these data show that a sequential expression of these lineage predominant TFs can play a critical role in defining the fate of CFs.

Driven by this hypothesis, we successfully differentiated human iPSCs into CFs by modifying a protocol initially developed to generate iPSC-EPCs. Importantly, we demonstrated that our human iPSC-CFs can serve as a faithful in vitro cell model to explore the underlying mechanisms of cardiac fibrosis and to screen anti-fibrotic drugs due to the following advantages. First, human iPSC-CFs highly resemble primary cells at the genetic and functional levels, and preserve a quiescent phenotype until passage 4. Therefore, the derived cells are sensitive to pro-fibrotic stimuli. Indeed, using this powerful in vitro platform, we demonstrated that human iPSC-CFs are highly responsive to both anti- and pro-fibrotic drugs. Second, human iPSCs can be used as an unlimited cell source to generate a large quantity of CFs for mechanistic studies and high throughput anti-fibrotic drug discovery. Third, the induction protocol for human iPSC-CF developed in this study will allow for testing of sex- and patient-specific cellular responses to candidate anti-fibrotic drugs in vitro.

A substantial body of evidence shows that crosstalk between fibroblasts and inflammatory cells regulates fibrosis in different injured tissues, such as the heart, lungs, and liver. Interestingly, using the complementary receptor-ligand analysis tool, intercellular communication networks generated using mouse scRNA-seq data suggest that fibroblasts reciprocally interact with a wide range of cell types other than inflammatory cells to regulate their cellular and biological functions. By coculturing human iPSC-CMs and iPSC-CFs, we identified that crosstalk between these two cell types through the ANP/BNP-NPR1 pathway may play a fundamental role in suppressing cardiac fibrosis. Importantly, we further showed that the anti-fibrotic effect seen in co-culture could be significantly enhanced by treating these cells with sacubitril, a drug that inhibits the activity of ANP/BNP-degrading enzyme neprilysin. Sacubitril has been used in combination with an angiotensin II type 1 receptor blocker called valsartan (a drug named LCZ696) to reduce the blood pressure and improve mortality in patients with heart failure. However, evidence shows that LCZ696 improved isoproterenol-induced cardiac fibrosis more significantly than valsartan alone in rats, suggesting the anti-fibrotic effect of neprilysin inhibitor beyond its diuretic function. In agreement with the animal study, a recent large-scale prospective cohort study showed that compared with baseline, the administration of LCZ696 for eight months drastically attenuated profibrotic signaling in heart failure patients with reduced ejection fraction.

Collectively, these results show that modulating fibrosis-related intercellular signaling cascades such as the ANP/BNP-NPR1 signaling pathway provides a promising route for antifibrotic therapies in addition to suppressing fibroblast activation directly.

In conclusion, the heterogeneity of fibroblasts in different tissues is associated with the expression of tissue-specific signature genes during development. The induction protocol for bona fide quiescent human iPSC-CFs developed in this study allowed us to identify and validate signaling pathways that may be involved in cardiac fibrosis. This study also sheds light on the significance of using human iPSC-CFs as a robust cell platform to test the efficacy of candidate anti-fibrotic drugs in a patient-specific manner.

### Methods

Human primary skin, cardiac, lung, bladder fibroblast, and brain pericyte cultures. Human primary cardiac fibroblasts were isolated and cultured as follows: a piece of fresh right atrial biopsy was weighed, minced into small pieces (1-2 mm³), and digested in 6-well peri-dishes as described. Human primary cardiac fibroblasts were grown and maintained in a fibroblast growth medium (C-23025, Promocell) supplemented with 1% penicillin-streptomycin (15140-122, Gibco) in a humidified atmosphere (95% air, 5% CO2) at 37° C. The medium was renewed every 2-3 days. Cells were passaged using standard trypsinization techniques at 80-90% confluency. The same protocol was also used to isolate and culture fibroblasts from human skin and lung tissues. Primary bladder fibroblasts were purchased from Lifeline (FC-0050) and maintained in the same fibroblast growth medium. Human brain pericytes were purchased from Cell systems (ACBRI 498) and cultured in SmGM-2 medium (Lonza, CC-3182). All experiments were carried out using cells at low cell passages (<P4) and cells were serum-starved for 24 hrs before treatment.

Quantitative real-time PCR. Total RNA was isolated using the Qiagen miRNeasy Mini Kit (Qiagen Sciences, Inc, Germantown, Md.) following the manufacturer's instruction. For reverse transcription, a High Capacity RNA-to-cDNA kit (Life Technologies) was used, and the cDNA template was synthesized based on 1 μg of total mRNA. After mixing 0.2 μl of the cDNA template, 1 μl of TaqMan® primer sets (Life Technologies), 10 μl of TaqMan® Master Mix (Life Technologies), and 8.8 μl of ddH2O in the reaction system, real-time quantitative PCR was performed on the StepOne™ Real-Time PCR System (Life Technologies). Each reaction was run in triplicate. Expression values were normalized to the average expression of GAPDH.

Immunoblotting and quantification. Cultured cells were homogenized in the RIPA buffer with proteinase and phosphatase inhibitor cocktails (78440, ThermoFisher), and quantified by Pierce™ BCA Protein Assay Kit (Pierce Biotechnology Inc.). A total of 20 μg protein was separated on NuPAGE® Novex® 4-12% Bis-Tris Protein Gels (Invitrogen, Carlsbad, Calif.). After being transferred to Amersham™ Hybond™ Blotting Membranes (GE Healthcare), proteins were incubated with primary antibodies against α-SMA (ab7817, Abcam), periostin (ab14041, Abcam), collagen Ill (ab7778, Abcam), fibronectin (ab6328, Abcam), MYH7 (sc-53089, Santa Cruz), and GAPDH (MA5-15738, Invitrogen). Band intensities for target bands were analyzed and quantified by the ImageJ Fuji program.

Immunofluorescent staining. Cells were fixed using 4% paraformaldehyde for 15 min, permeabilized with 0.2% Triton X-100 in PBS (Sigma-Aldrich) for 10 min, and blocked with 3% bovine serum albumin (BSA). Cells were incubated overnight at 4° C. with primary antibody or isotype controls diluted in 3% BSA. Cells were incubated with Alexa Fluor™ secondary antibodies (1:500 dilution) at room temperature for 1 hr, and mounted with mounting medium with 4′,6-diamidino-2-phenylindole (DAPI) (Vector Laboratories). Primary antibodies against NANOG2 (ab109250, Abcam), SSEA-4 (ab16287, Abcam), NKX2.5 (MAB2444, R&D), WT1 (ab89901, Abcam), ZO1 (40-2200, Invitrogen), GATA4 (sc-25310, Santa Cruz), DDR2 (sc-81707, Santa Cruz), COLI (ab90395, Abcam), VIM (V6630, Sigma-Aldrich), POSTN (ab14041, Abcam), TBX20 (hpa008192, Sigma-Aldrich), TCF21 (hpa013189, Sigma-Aldrich), α-SMA (ab7817, Abcam), CNN1 (ab46794, Abcam), SMTN (ab204305, Abcam), and TAGLN (ab14106, Abcam) were used. Labeled cells were imaged by a confocal microscope (Carl Zeiss, LSM 510 Meta, Gottingen, Germany).

Fluorescence-activated cell sorting (FACS) analysis. Cells were dissociated into singlets by TrypLE™ (12605, Gibco) for 10 min at 37° C., and the cell suspension was filtered through a 40-μm cell strainer (BD Falcon, San Diego) to remove cell clumps. Single cells were fixed and permeabilized by BD CytofixCytoperm™ buffer (BD 554722) for 20 min at 4° C. Next, cells were incubated with primary antibodies followed by Alexa Fluor™ secondary antibodies (Invitrogen). Primary antibodies against COLI (ab90395, Abcam), DDR2 (sc-81707, Santa Cruz), TCF21 (hpa013189, Sigma-Aldrich), α-SMA (ab7817, Abcam), and WT1 (ab89901, Abcam) were used. The percentage of fibroblasts was calculated as the ratio of TCF21+, DDR2+, and COL-1+ cells in the whole population, respectively. The percentage of myofibroblasts was calculated as the ratio of α-SMA+ cells in the whole population.

Differentiation of human induced pluripotent stem cell-derived cardiomyocytes. All of the protocols for this study were approved by the Stanford University Human Subjects Research Institutional Review Board (IRB). Human induced pluripotent stem cell-derived cardiomyocyte (iPSC-CM) differentiation protocol was used as previously described. Briefly, human iPSC lines were maintained on Matrigel-coated plates (CB-40234, BD Biosciences, San Jose, Calif.) in Essential 8™ Medium (A1517001, Gibco®, Life Technologies). Human iPSCs were treated with 6 μM of CHIR99021 (S2924, Selleck chemicals) for 2 days, recovered in RPMI medium (11875-119, Gibco®, Life Technologies) with B27 supplements minus insulin (A1895601, Gibco®, Life Technologies) (RPMI+B27-insulin) for 24 hrs, treated with 5 μM of IWR-1 (10161, Sigma) for 2 days, and then placed in RPMI+B27-insulin for another 2 days, and finally switched to RPMI medium with B27 supplements (17504044, Gibco®, Life Technologies). Beating cells were observed at days 9-11 after differentiation. Human iPSC-CMs were re-plated and purified with the glucose-free RPMI medium (11879020, Gibco®, Life Technologies) for 2-3 rounds. Typically, the purity of human iPSC-CMs (TNNT2+) is >90% after purification. Cultures were maintained in a humidified atmosphere (95% air, 5% CO2) at 37° C.

Differentiation of human iPSC-derived cardiac fibroblasts. Human iPSCs were treated with 6 μM of CHIR99021 for 2 days, recovered in RPMI+B27-insulin for 24 hrs, treated with 5 μM of IWR-1 (10161, Sigma) for 2 days, and then RPMI+B27-insulin for another 24 hrs. On day 6, human induced pluripotent stem cell-derived cardiac progenitor cells (iPSC-CPCs) were re-plated at a density of 20,000 cells/cm2 in advanced DMEM medium (12634028, Gibco®, Life Technologies). On day 7 or 8, cells were treated with 5 μM of CHIR99021 and 2 μM of retinoic acid (R2625, Sigma-Aldrich) for 2 days, and recovered in advanced DMEM for 3 days. Typically, the purity of human induced pluripotent stem cell-derived epicardial cells (iPSC-EPCs) is >90% (WT1+). Human iPSC-EPCs were re-plated and treated with 10 μM of FGF2 (100-18B, PeproTech) and 10 μM of SB431542 (S1067, Selleck chemicals) in a fibroblast growth medium for another 6 days. >90% purity of iPSC-CFs (COL-I+/DDR2+/TCF21+) can be achieved as assessed by FACS. Cultures were maintained in a humidified atmosphere (95% air, 5% CO2) at 37° C.

Differentiation of human iPSC-derived cardiac smooth muscle cells. Human iPSC-EPCs were plated in a chemically defined medium and treated with PDGF-BB (10 ng/ml, 100-14B, PeproTech) and TGFβ1 (2 ng/ml, 100-2, Peprotech) for 12 days. 3 Over 90% purity of human induced pluripotent stem cell-derived smooth muscle cells (iPSC-SMCs) (SMTN+/TAGLN+) can be achieved as assessed by immunofluorescent staining. Cultures were maintained in a humidified atmosphere (95% air, 5% CO2) at 37° C.

Drug treatment. Unless otherwise stated, cells were treated with all drugs for 48 hrs. We used 5 ng/ml of TGFβ1 (240-B-010, R&D) to activate iPSC-CFs. Due to the short half-life, BNP (CYT-369, Prospec) was added into the medium three times a day. We used 20 μM of sacubitril (SML1380, Sigma) to inhibit ANP/BNP degradation. We used pirfenidone (S2907, Selleck Chemicals) and doxorubicin (25316-40-9, Selleck Chemicals) to test their anti-fibrotic and pro-fibrotic effects on iPSC-CFs, respectively.

Indirect and direct contact co-culture. Transwells (353102, Corning) containing fibroblasts were suspended over the iPSC-CMs in the plastic bottom dish, so that the base of the Transwell sat within the culture medium on the iPSC-CMs but did not touch the base of the dish. As a control, iPSC-CMs were co-cultured with iPSC-CMs in the transwells. The co-cultures were incubated at 37° C. and 5% CO2 before analysis. For direct contact co-culture, we first labeled fibroblasts and iPSC-CMs with CFSE (C34554, Thermo Fischer) and Far cell trace red (C34564, Thermofisher) dyes, respectively. Then, iPSC-CFs were seeded on top of the iPSC-CM monolayer at a ratio of 1:5, allowing for direct cell-cell contact. After 5 days of co-culture, the two cell types were separated by FACS sorting for downstream molecular analyses. The medium used for co-culture is the mixture of RPMI+B27 and fibroblast growth medium at a ratio of 3:1.

Colorimetric assays. The cells were treated in the absence or presence of TGF-β for 48 hrs. Quantification of total secreted collagen in the cell culture supernatant was performed using a Sirius red collagen detection kit (9062, Chondrex). The colorimetric assay was performed according to the manufacturer's protocol.

Gel contraction assay. Collagen gel contraction assays were performed in three independent lines of iPSC-CFs (2.0×105 cells per well) using a Cell Contraction Assay kit (CBA5020, Cell Biolabs) as per the manufacturer's protocol. The cells were treated in the absence or presence of TGF-β±a TGF-β inhibitor SB431542 (SB). The gels were imaged after 24 hrs and gel area was quantified using ImageJ software (version 1.49).

In vitro wound-healing assay. The migration of human iPSC-CFs and primary CFs was determined using a Cytoselect™ 24-Well Wound Healing Assay Kit (CBA-120, Cell Biolabs). Briefly, after confluent monolayers of CFs were formed, cells were synchronized in a low serum medium (0.5% FBS) for 24 hrs. After the wound-generating scaffold was removed from each well, cells were washed in PBS to remove residual cells in the wounded area. Cells were treated with vehicle or PDGF-BB (25 ng/ml), and images were captured using a light microscope in identical areas at baseline, 12 hrs, and 24 hrs post-treatment. The cell-free areas at each time point were measured by Image-Pro Plus (version 6.0, Media Cybernetics). For quantification purposes, the wound area generated at 0 hr was set as S0, the uncovered areas captured at 12 hrs, and 24 hrs were recorded as Si (i=12 or 24). The wound closure rate was then calculated by the following equation: wound closure rate

$$( \% ) = \frac{( {S}_{0} - {S}_{i )}}{{S}_{0}} \times 100.$$

Bioinformatics analysis. We downloaded all single-cell RNA sequencing (scRNA-seq) data from the Tabula Muris database. Specifically, Seurat objects were downloaded from Figshare and cell annotations were downloaded from the Tabula Muris Github website. We filtered out cells with fewer than 500 genes expressed or 50,000 reads covered with “FilterCells” function. Next, raw counts data were normalized using “Normalize Data” function and further regressed against the number of total reads (“nRead”) and mitochondrial proportion (“percent.mito). The dimensional reduction was performed on genes with dispersion >0.5 using Principal Component Analysis (PCA) algorithm implemented in “RunPCA” function. We ran t-distributed stochastic neighbor embedding (t-SNE) algorithm using top 20 PCs for all FB and top 10 PCs for heat FB with “RunTSNE” function. We detected differentially expressed genes (DEGs) among organs and between sexes using the Wilcoxon rank sum test under adjusted P-value<0.01, fold-change ≥2, and >25% cells expressed in either group, and only positive markers were reported. All the above steps were performed with Seurat R package. 5 We performed pathway enrichment analysis on DEGs with a hypergeometric test using geneAnswers R package under FDR<0.05 and visualized the results using Cytoscape software v3.6.6 The ligand-receptor analysis was performed following the steps previously and visualized with Cytoscape software.

Statistical analyses. For statistical analyses, the Student's t-test was used to compare two normally distributed data sets. One-way or two-way ANOVA was used, where appropriate, to compare multiple data sets, and Bonferroni multiple comparison after-tests were used for all pairwise comparisons, depending on the properties of the data sets. A P value<0.05 was considered to be statistically significant. All data are shown as mean±SEM.

### REFERENCES

Tomasek J J, Gabbiani G, Hinz B, Chaponnier C and Brown R A. Myofibroblasts and mechano-regulation of connective tissue remodelling. Nat Rev Mol Cell Biol. 2002:3:349-63.
Zeisberg M and Kalluri R. Cellular mechanisms of tissue fibrosis. 1. Common and organ specific mechanisms associated with tissue fibrosis. Am J Physiol Cell Physiol. 2013; 304:C216-25.
Paik D T, Tian L. Lee J, Sayed N, Chen I Y, Rhee S, Rhee J W, Kim Y, Wirka R C, Buikema J W, Wu S M, Red-Horse K. Quertermous T and Wu J C. Large-scale single-cell RNA-seq reveals molecular signatures of heterogeneous populations of human induced pluripotent stem cell-derived endothelial cells. Circ Res. 2018; 123:443-450.
Shi Y, Inoue H, Wu J C and Yamanaka S. Induced pluripotent stem cell technology: a decade of progress. Nat Rev Drug Discov. 2017; 16:115-130.
The Tabula Muris Consortium. Single-cell transcriptomics of 20 mouse organs creates a Tabula Muris. Nature. 2018; 562:367-372.
Graf T and Enver T. Forcing cells to change lineages. Nature. 2009; 462:587-94.
Xu J, Du Y and Deng H. Direct lineage reprogramming: strategies, mechanisms, and applications. Cell Stem Cell. 2015; 16:119-34.
Singh M K, Christoffels V M, Dias J M, Trowe M O, Petry M, Schuster-Gossler K. Burger A, Ericson J and Kispert A. Tbx20 is essential for cardiac chamber differentiation and repression of Tbx2. Development (Cambridge, England). 2005; 132:2697-707.
Molkentin J D, Lin Q, Duncan S A and Olson E N. Requirement of the transcription factor GATA4 for heart tube formation and ventral morphogenesis. Genes & Development. 1997; 11:1061-72.
Tandon P, Miteva Y V, Kuchenbrod L M, Cristea I M and Conlon F L. Tcf21 regulates the specification and maturation of proepicardial cells. Development. 2013; 140:2409-21.
Iyer D, Gambardella L, Bernard W G, Serrano F, Mascetti V L, Pedersen R A, Sinha S and Talasila A. Robust derivation of epicardium and its differentiated smooth muscle cell progeny from human pluripotent stem cells. Development. 2016; 143:904.
Acharya A, Baek S T, Huang G, Eskiocak B, Goetsch S, Sung C Y, Banfi S, Sauer M F, Olsen G S, Duffield J S, Olson E N and Tallquist M D. The bHLH transcription factor Tcf21 is required for lineage-specific EMT of cardiac fibroblast progenitors. Development. 2012; 139:2139-49.
Iyer D, Gambardella L, Bernard W G, Serrano F, Mascetti V L, Pedersen R A, Talasila A and Sinha S. Robust derivation of epicardium and its differentiated smooth muscle cell progeny from human pluripotent stem cells. Development. 2015; 142:1528-41.
Armulik A, Genove G and Betsholtz C. Pericytes: developmental, physiological, and pathological perspectives, problems, and promises. Developmental Cell. 2011:21:193-215.
Wang Z, Wang D Z, Hockemeyer D, McAnally J, Nordheim A and Olson E N. Myocardin and ternary complex factors compete for SRF to control smooth muscle gene expression. Nature. 2004; 428:185-9.
Liu Y, Sinha S, McDonald O G, Shang Y, Hoofnagle M H and Owens G K. Kruppel-like factor 4 abrogates myocardin-induced activation of smooth muscle gene expression. The Journal of Biological Chemistry. 2005; 280:9719-27.
Schafer et al. IL-11 is a crucial determinant of cardiovascular fibrosis. Nature. 2017; 552:110-115.
Furtado et al. Cardiogenic genes expressed in cardiac fibroblasts contribute to heart development and repair. Circ Res. 2014; 114:1422-34.
Pandya K, Kim H S and Smithies O. Fibrosis, not cell size, delineates beta-myosin heavy chain reexpression during cardiac hypertrophy and normal aging in vivo. Proceedings of the National Academy of Sciences of the United States of America. 2006; 103:16864-9.
Swaney J S, Roth D M, Olson E R, Naugle J E, Meszaros J G and Insel P A. Inhibition of cardiac myofibroblast formation and collagen synthesis by activation and overexpression of adenylyl cyclase. Proceedings of the National Academy of Sciences of the United States of America. 2005:102:437-42.
Fan D, Takawale A, Lee J and Kassir Z. Cardiac fibroblasts, fibrosis and extracellular matrix remodeling in heart disease. Fibrogenesis & Tissue Repair. 2012; 5:15.
Wang Z, Juttermann R and Soloway P D. TIMP-2 is required for efficient activation of proMMP-2 in vivo. The Journal of Biological Chemistry. 2000; 275:26411-5.
Taniguchi H. Ebina M, Kondoh Y, Ogura T, Azuma A, Suga M, Taguchi Y, Takahashi H, Nakata K. Sato A, Takeuchi M, Raghu G, Kudoh S and Nukiwa T. Pirfenidone in idiopathic pulmonary fibrosis. The European Respiratory Journal. 2010; 35:821-9.
Levick S P, Soto-Pantoja D R, Bi J, Hundley W G, Widiapradja A. Manteufel E J, Bradshaw T W and Melendez G C. Doxorubicin-induced myocardial fibrosis involves the Neurokinin-1 receptor and direct effects on cardiac fibroblasts. Heart, lung & circulation. 2018.
Howard C M and Baudino T A. Dynamic cell-cell and cell-ECM interactions in the heart. J Mol Cell Cardiol. 2014; 70:19-26.
Kerkela R, Ulvila J and Magga J. Natriuretic peptides in the regulation of cardiovascular physiology and metabolic events. J Am Heart Assoc. 2015; 4:e002423.
Potter L R. Natriuretic peptide metabolism, clearance and degradation. The FEBS journal. 2011; 278:1808-17.
Singh J S S, Burrell L M, Cherif M, Squire I B, Clark A L and Lang C C. Sacubitril/valsartan: beyond natriuretic peptides. Heart. 2017; 103:1569-1577.
Horie M, Miyashita N, Mikami Y, Noguchi S, Yamauchi Y, Suzukawa M, Fukami T, Ohta K, Asano Y, Sato S, Yamaguchi Y, Ohshima M, Suzuki H I, Saito A and Nagase T. TBX4 is involved in the super-enhancer-driven transcriptional programs underlying features specific to lung fibroblasts. Am J Physiol Lung Cell Mol Physiol. 2018; 314:L177-L191.
Weiskirchen R, Weiskirchen S and Tacke F. Organ and tissue fibrosis: Molecular signals, cellular mechanisms and translational implications. Mol Aspects Med. 2018.
Whyte W A, Orlando D A, Hnisz D, Abraham B J, Lin C Y, Kagey M H, Rahl P B, Lee T I and Young R A. Master transcription factors and mediator establish super-enhancers at key cell identity genes. Cell. 2013; 153:307-19.
Watt A J, Battle M A, Li J and Duncan S A. GATA4 is essential for formation of the proepicardium and regulates cardiogenesis. Proceedings of the National Academy of Sciences. 2004; 101:12573-12578.21
Yan X, Zhang H, Fan Q, Hu J, Tao R. Chen Q, Iwakura Y, Shen W, Lu L, Zhang Q and Zhang R. Dectin-2 deficiency modulates Th1 differentiation and improves wound healing after myocardial infarction. Circ Res. 2017; 120:1116-1129.
Hartupee J and Mann D L. Role of inflammatory cells in fibroblast activation. J Mol Cell Cardiol. 2016; 93:143-8.
Karlmark K R, Weiskirchen R, Zimmermann H W, Gassler N, Ginhoux F, Weber C, Merad M, Luedde T, Trautwein C and Tacke F. Hepatic recruitment of the inflammatory Gr1+ monocyte subset upon liver injury promotes hepatic fibrosis. Hepatology. 2009:50:261-74.
Miyoshi T, Nakamura K, Miura D, Yoshida M, Saito Y, Akagi S, Ohno Y, Kondo M and Ito H. Effect of LCZ696, a dual angiotensin receptor neprilysin inhibitor, on isoproterenol-induced cardiac hypertrophy, fibrosis, and hemodynamic change in rats. Cardiology Journal. 2018.
Zile M R, O'Meara E. Claggett B, Prescott M F, Solomon S D, Swedberg K. Packer M, McMurray J J V, Shi V, Lefkowitz M and Rouleau J. Effects of Sacubitril/Valsartan on biomarkers of extracellular matrix regulation in patients With HFrEF. J Am Coll Cardiol. 2019:73:795-806.

### Example 2

### Differentiation Protocol

By comparing the single-cell transcriptome profiles from various organs, we previously identified distinct tissue-specific genes in cardiac fibroblasts, including early cardiac development-related genes (GATA4, TBX20, MEF2C, HAND2, and GATA6) and epicardial markers such as TCF21, indicating CFs at large are derived from cardiac and epicardial lineages. Based on that, we are able to show that human iPSC-CFs are successfully generated after the sequential differentiation of intermediate cardiac progenitor cells and epicardial cells.

1. Culture human iPSCs on Matrigel-coated 6-well plates in E8 medium as previously reported. 2. On day 0, prepare RPMI/B27 minus insulin (RPMI/B27-insulin) medium containing 6 μM of CHIR99021 (CHIR, GSK3 inhibitor). Once the cell confluence reaches 80-90%, aspirate spent medium and add 3 ml of the newly prepared RPMI/B27-insulin+CHIR medium to each well of the 6-well plate. Leave cells undisturbed for 2 days (see Note 1). 3. On day 2, aspirate the spent medium from each well of 6-well plates and add 3 ml of RPMI/B27-insulin medium. 4. On day 3, prepare RPMI/B27-insulin medium containing 5 μM of IWR-1 (Wnt inhibitor). Aspirate the spent medium and then add 3 ml of the freshly prepared RPMI/B27-insulin+IWR-1 medium to each well of 6-well plates. 5. On day 5, aspirate the spent medium from each well of 6-well plates and add 3 ml of RPMI/B27-insulin medium (see Note 2).

Differentiation of Cardiac Progenitors into Proepicardial Cells. 1. On day 6, prepare Advanced DMEM/GlutaMAX medium containing 5 μM of CHIR99021, 2 μM of retinoid acid, 5 μM of Y27632 (ROCK inhibitor), and 1% FBS. 2. Aspirate the spent medium, add 1 ml of Accutase per well in a 6-well plate, and incubate the plate in a 37° C., 5% CO₂ incubator for 5 min. Gently pipette 5-10 times to singularize the cells, and then transfer the cell mixture to a 15-ml conical tube containing 1 ml of RPMI medium. Count the cell density with a hemocytometer, centrifuge the cells at 200×g for 3 min at room temperature, and aspirate the supernatant. 3. Resuspend the cell pellet in medium prepared in step 6, and then seed singularized cells onto a Matrigel-coated cell culture dish at a density of 20,000 per cm² (or a 1:12 split ratio). Incubate the plate at 37 99_C, 5% CO2 overnight to allow cell attachment. 4. On day 7, aspirate the spent medium and add 2 ml of Advanced DMEM/GlutaMAXmedium containing 5 μM of CHIR99021 and 2 μM of retinoid acid per well. 5. On day 9, aspirate the spent medium and add 2 ml of Advanced DMEM/GlutaMAX medium per well. 6. On day 11, prepare Advanced DMEM/GlutaMAX medium containing 2 μM SB431542 (a TGFβ inhibitor). 7. Aspirate the spent medium, add 1 ml Accutase per well, and incubate the plate in a 37 109_C, 5% CO2 incubator for 5 min. Pipette 5-10 times to singularize the cells, and then transfer the cell mixture to a 15-ml conical tube containing 1 ml of Advanced DMEM/GlutaMAX medium. Centrifuge the cells at 200×g for 3 min and aspirate the supernatant. 8. Resuspend the cell pellet in Advanced DMEM/GlutaMAX medium containing 2 μM SB431542 (prepared in step 11), and seed cells onto a Matrigel-coated cell culture dish at a split ratio of 1:3 to 1:6. Incubate the plate at 37° C., 5% CO₂ overnight to allow cell attachment. 9. On day 12 and every other day thereafter, change the medium with Advanced DMEM/GlutaMAX containing 2 μM of SB431542. 10. Once the cells are confluent, the proepicardial cells can be passaged for long-term maintenance, frozen down, or further differentiated into CFs (see Note 3).

Differentiation of Quiescent Cardiac Fibroblasts from iPSC Derived Epicardial Cells. 1. On day 14, prepare fibroblast growth medium containing 20 ng/ml of FGF2 and 10 μM of SB431542. 2. Aspirate the spent medium, add 1 ml of Accutase per well in a 6-well plate, and incubate the plate in a 37 129_C, 5% CO2 incubator for 5 min. Pipette 5-10 times to singularize the cells, and then transfer the cell suspension to a 15-ml conical tube containing 1 ml of Advanced DMEM/GlutaMAX medium. Count the cell density with a hemocytometer, centrifuge the cells at 200×g for 3 min at room temperature, and aspirate the supernatant. 3. Resuspend the epicardial cells and seed them onto a Matrigel-coated cell culture dish at a density of 10,000 cells per cm² (or a 1:3 split ratio) in the medium prepared in step 16. Incubate the plate at 37° C., 5% CO2 overnight to allow cell attachment. 4. On day 16, change the spent medium with 2 ml freshly prepared fibroblast growth medium with 20 ng/ml of FGF2 and 10 μM of SB431542. 5. On day 18, resuspend the cells and seed them onto a Matrigel-coated cell culture dish at a density of 10,000 cells per cm² (or a 1:3 split ratio) in fibroblast growth medium containing 20 ng/ml of FGF2 and 10 μM of SB431542. 6. On day 20 and each other day thereafter, aspirate the medium from each well of the 6-well plate, and add 2 ml per well of fibroblast growth medium with 10 μM of SB431542. The quiescent CFs (FIG. 2) can be passaged, frozen down for long-term storage, or collected for characterization (see Note 4).

Notes. 1. In general, culturing iPSCs in 6 μM of CHIR99021-containing medium for 48 h is sufficient to initiate cardiac progenitor cell differentiation. However, if massive cell death or detachment is observed on day 2, the concentration (4-8 μM) and duration of CHIR99021 (24-48 h) should be titrated for your specific iPSC lines. 2. As both iPSC-derived cardiomyocytes and CFs are generated from the cardiac progenitor cells, differentiation steps during the first 6 days are identical for both cell types. If the cardiac progenitor cells are maintained in original plates on day 6, robust beating sheets of cTnT⁺ cardiomyocytes will be observed, which suggests that cell density toggles epicardial vs. cardiomyocyte differentiation. 3. This protocol is developed based on an earlier protocol for iPSC-epicardial cell differentiation. A low yield of CFs may be due to poor quality of epicardial cells. Typical epicardial cells should have a cobblestone-like morphology, and the purity of WT1+ cells should be >90% on day 12. 4. Fibroblasts can undergo spontaneous transdifferentiation into myofibroblasts during in vitro culture conditions. The majority of iPSC-derived CFs can preserve a quiescent state (˜5% αSMA+ cells) until passage 5 with the presence of TGFβ inhibitor SB431542 in fibroblast growth medium. The TGFβ inhibitor should be removed from the medium at least 1 day prior to transdifferentiation experiments.

References: Hinderer S, Schenke-Layland K (2019) Cardiac fibrosis—a short review of causes and therapeutic strategies. Adv Drug Deliv Rev 146:77-82. 2. Zeisberg M, Kalluri R (2013) Cellular mechanisms of tissue fibrosis. 1. Common and organ-specific mechanisms associated with tissue fibrosis. Am J Physiol Cell Physiol 304(3):C216-C225. 3. Furtado M B, Costa M W, Pranoto E A, Salimova E, Pinto A R, Lam N T, Park A, Snider P, Chandran A. Harvey R P, Boyd R, Conway S J, Pearson J, Kaye D M, Rosenthal N A (2014) Cardiogenic genes expressed in cardiac fibroblasts contribute to heart development and repair. Circ Res 114(9):1422-1434. 4. Shi Y, Inoue H, Wu J C, Yamanaka S (2017) Induced pluripotent stem cell technology: a decade of progress. Nat Rev Drug Discov 16 (2):115-130 5. Zhang H, Tian L, Shen M, Tu C, Wu H, Gu M, Paik D T, Wu J C (2019) Generation of quiescent cardiac fibroblasts from human induced pluripotent stem cells for in vitro modeling of cardiac fibrosis. Circ Res 125(5):552-566. 6. (2018) Single-cell transcriptomics of 20 mouse organs creates a Tabula Muris. Nature 562 (7727):367-372. 7. Burridge P W, Matsa E, Shukla P. Lin Z C, Churko J M, Ebert A D, Lan F, Diecke S, Huber B, Mordwinkin N M, Plews J R, Abilez O J, Cui B, Gold J D, Wu J C (2014) Chemically defined generation of human cardiomyocytes. Nat Methods 11(8):855-860 8. Bao X, Lian X, Hacker T A, Schmuck E G, Qian T, Bhute V J, Han T, Shi M, Drowley L, Plowright A, Wang Q D, Goumans M J, Palecek S P (2016) Long-term self-renewing human epicardial cells generated from pluripotent stem cells under defined xeno-free conditions. Nat Biomed Eng 1:0003. 9. Swaney J S, Roth D M, Olson E R, Naugle J E, Meszaros J G, Insel P A (2005) Inhibition of cardiac myofibroblast formation and collagen synthesis by activation and overexpression of adenylyl cyclase. Proc Natl Acad Sci USA 102 (2):437-442

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. An in vitro culture method of generating a population of human cardiac fibroblast cells from a human pluripotent stem cell, the method comprising:
(a) generating in culture a population of human cardiac progenitor cells from human pluripotent stem cells;
(b) generating in culture a population of human epicardial cells from the human cardiac progenitor cells;
(c) generating in culture a population of human cardiac fibroblast cells from the human epicardial cells by culture in the presence of an effective concentration of an inhibitor of transforming growth factor β (TGF-β).

2. The method of claim 1, wherein the population of human cardiac fibroblast cells is at least 80% pure.

3. The method of claim 1, wherein the population of human cardiac fibroblast cells is at least 90% pure.

4. The method of claim 1, wherein the human cardiac fibroblast cells are quiescent.

5. The method of claim 1, wherein step (c) comprises culturing the population of human epicardial cells in the presence of an effective concentration of an FGF agonist, and a TGRβ inhibitor, for a period of from 5 to 8 days.

6. The method of claim 5 wherein the effective concentration of an FGF agonist, and a TGRβ inhibitor, is each from about 1 mM to about 50 mM.

7. The method of claim 1, wherein step (a) comprises culturing a population of human induced pluripotent stem cells in the presence of an effective concentration of a Wnt agonist for a period of from about 24 to about 72 hours; culturing in medium for a period of from about 12 to about 38 hours; culturing in the presence of an effective concentration of a Wnt inhibitor for a period of from about 24 to about 72 hour; and culturing in medium for a period of from about 12 to about 36 hours to generate the population of cardiac progenitor cells.

8. The method of claim 1, wherein step (b) comprises culturing the population of cardiac progenitor cells in the presence of an effective concentration of a Wnt agonist, and retinoic acid, for a period of from about 24 to about 72 hours.

9. A population of human cardiac fibroblast cells generated by the method of claim 1.

10. A co-culture comprising the population of cardiac fibroblast cells of claim 9; and a one or more additional cardiac cell types.

11. The co-culture of claim 10, comprising one or both of cardiac smooth muscle cells; and cardiac myocytes.

12. A method for screening of a candidate agent, the method comprising: contacting the candidate agent with one or a panel of quiescent human cardiac fibroblasts of claim 9, or a co-culture; and determining the effect of the agent on morphologic, genetic or functional parameters.

13. The method of claim 12, wherein the phenotype is fibrosis.

14. The method of claim 12, wherein a panel of human cardiac fibroblasts comprises at least 2 differing genotypes.

15. The method of claim 14, at least one genotype comprises an allele associated with cardiac disease.

16. The method of claim 12, wherein determining the effect of the agent comprises single cell analysis.

17. The method of claim 12, wherein the candidate agent is a drug candidate.

18. The method of claim 12, wherein the candidate agent is a genetic agent.
